(19) United States
(12) Patent Application Publication
(10) Pub. No.: US 2009/0196894 A1
(43) Pub. Date: Aug. 6, 2009

(54) Self Tanning Composition

(76) Inventors: Thomas Ehlis, Freiburg (DE); Stefan Müller, Weil am Rhein (DE); Sebastien Mongiat, Sierentz (FR)

Correspondence Address: JoAnn Villamizar;Ciba Corporation/Patent Department, 540 White Plains Road, P.O. Box 2005, Tarrytown, NY, 10591, US

(21) Appl. No.: 12/227,769

(22) PCT Filed: May 21, 2007
(86) PCT No.: PCT/EP2007/054864

§ 371 Date: Mar. 20, 2009

(30) Foreign Application Priority Data
May 31, 2006 (EP) 06114757.5

(51) Int. Cl. A61K 9/14; A61K 8/49

(52) U.S. Cl. 424/401; 424/59; 424/63; 424/70.6

(57) ABSTRACT

Disclosed is a method for the colouration of skin or a keratinic fibre, characterized in that the skin or keratinic fibre is contacted with a formulation containing a dispersion of solid particles of a tanning substance selected from a compound of the formula (I) wherein i is 0 or 1 and each of R₁, R₂, R₃, R₄, R₅, R₆, R₇, R₈ independently of each other is selected from group consisting of hydrogen, OH, C₁-C₆alkyl, C₁-C₆hydroxyalkyl and C₂-C₆al-kenyl; in an organic liquid of low polarity or non-polar organic liquid.

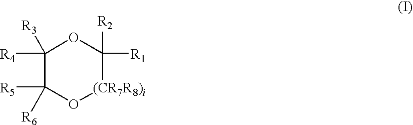

The present invention relates to a method for the artificial tanning of the skin or to the colouration of keratinic fibres such as hair by a solid particle dispersion of a tanning substance, to a corresponding use and composition, and to a process for the preparation of a cosmetic composition containing a solid particle dispersion of a tanning substance.

Skin self-tanning substances, which are also usable for coloration of other keratinic materials such as hair, are substances which produce brown dyes upon contact with the skin as a result of a chemical reaction with the keratin without the action of natural or artificial light (e.g. on reaction with primary or secondary amino group, Maillard reaction). They give brown-coloured substances e.g. in the stratum corneum of the skin. The reaction usually is complete after 4 to 12 hours; the tan cannot be washed off and is removed on the skin only with normal desquamation. It usually takes approximately 5 to 15 days until the skin is completely decoloured.

Typically, these substances are compounds containing aldol, ketol or dicarbonyl groups in the molecule, dihydroxyacetone (DHA) being the best known representative. Besides dihydroxyacetone, the following compounds are of importance: glyceraldehyde, 6-aldo-D-fructose, hydroxymethylglyoxal, mucondialdehyde, mucondialdehyde tetraethylacetal, malealdehyde, substituted succindialdehydes (see also EP-A-0 903 342), 2,2-dihydroxymethyl-1,3-dioxolane or substituted derivatives thereof (US-A-2003/108495).

However, the practical use of self-tanning active ingredients, for example DHA, in self-tanning compositions has disadvantages, such as inadequate stability and high reactivity with other formulation components (discoloration, formation of unpleasant odours), poor solubility in nonpolar organic solvents (virtually insoluble) and difficult formulatability.

Some proposals have been made to avoid direct application of the tanning substances, e.g. by applying precursors or derivatives of the active substances, which delay the tanning reaction until cleavage during application occurs (see for example EP-A-0 796 838 or WO-A-01/085124). U.S. Pat. No. 6,007,796 and WO-A-05/025531 propose encapsulated forms of the tanning substance.

It has now been found that certain active substances may be applied as solid particles to achieve good colouration or tanning effect with smooth and uniform colour generation, while cosmetic formulations containing these particles show a better stability than those containing the dissolved substances.

Main application field is the artificial tanning of the skin, the preparations of the invention may also be used for the levelling of differences in the brightness of the skin (e.g. in case of hyperpigmentation), for enhancing the natural sun protection factor of the skin, for the protection of the skin against electromagnetic radiation in the range of visible light (wavelength>400 nm) or near UV light (UV-A1, especially wavelengths>360 nm), and for the colouration of keratinic fibres such as human hair.

Thus, the present invention generally pertains to a method for the colouration of skin or a keratinic fibre, characterized in that the skin or keratinic fibre is contacted with a formulation containing a dispersion of solid particles of a tanning substance selected from a compound of the formula I

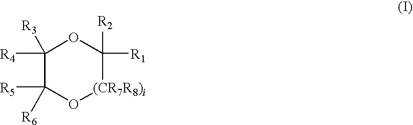

wherein i is 0 or 1 and each of R₁, R₂, R₃, R₄, R₅, R₆, R₇, R₈ independently of each other is selected from group consisting of hydrogen, OH, C₁-C₆alkyl, C₁-C₆hydroxyalkyl and C₂-C₆alkenyl; in an organic liquid of low polarity or non-polar organic liquid.

Preferably, the formulation does not contain chitin or chitosan; and does not contain a sulfite ion forming agent such for example sodium sulfite.

Most preferred are compounds of the formula II or III

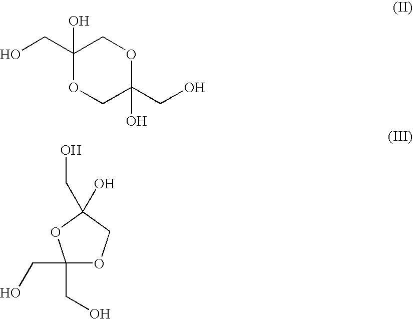

as well as isomers thereof, e.g. compounds of CA Reg. Nos. 62147-49-3 or 51795-26-7 or 675841-66-4 or 62182-56-3 or 757972-78-4.

Preferably, an organic liquid of low polarity or non-polar organic liquid is a silicon oil, a polysiloxane, a hydrocarbon-based oil or a fluoro oil or a mixture thereof. Of special interest are silicon oils, polysiloxanes, hydrocarbon-based oils or fluoro oils of high volatility.

As used herein, the term “hydrocarbon-based oil” means an oil mainly comprising hydrogen and carbon atoms, and possibly oxygen, nitrogen, sulfur and/or phosphorus atoms. The hydrocarbon-based oils may be chosen from hydrocarbon-based oils comprising from 8 to 16 carbon atoms, and for example branched C₈-C₁₆ alkanes, for instance C₈-C₁₆ isoalkanes of petroleum origin (also known as isoparaffins), for instance isododecane (also known as 2,2,4,4,6-pentamethylheptane), isohexadecane and, for example, the oils sold under the trade names Isopars® and Permethyls®.

Volatile silicon oils: for instance volatile linear or cyclic silicone oils, such as those with a viscosity ≦5 centistokes (5×10⁻⁶ m2/s) and for example comprising from 2 to 10 silicon atoms and from 2 to 7 silicon atoms, these silicones optionally comprising alkyl or alkoxy groups comprising from 1 to 10 carbon atoms. As volatile silicone oils that may be used in the disclosure, non-limiting mention may be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltri-siloxane, decamethyltetrasiloxane and dodecamethylpentasiloxane, and mixtures thereof.

Volatile fluoro-oils: non-limiting examples of volatile fluoro oils that may be mentioned include nonafluoroethoxybutane, nonafluoromethoxybutane, decafluoropentane, tetradecafluorohexane and dodecafluoropentane, and mixtures thereof.

The expression “volatile fluoro oil” should be understood as meaning an oil having, at 25° C., a saturating vapor pressure at least equal to 50 Pa. Suitable volatile fluoro oils which satisfy the above density and vapor pressure criteria include the following fluoro compounds:

1) perfluorocycloalkyls having formula I′

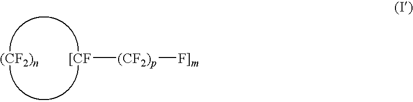

in which n is 4 or 5, m is 1 or 2, and p is 1, 2 or 3, with the proviso that, when m=2, the groups are not necessarily alpha to each other;
2) perfluoroalkanes of the formula II′

CF₃—(CF₂)ₘCF₂X (II′)

in which m is 2 to 8, and X represents Br or F;
3) fluoroalkyls or heterofluoroalkyls of the formula III′

CH₃—(CH₂)ₙ-[Zₜ]-(CF₂)ₘ—CF₃ (III′)

in which t is 0 or 1, n is 0, 1, 2 or 3, m is 2, 3, 4 or 5, and Z is 0, S or NR, wherein R is hydrogen or a —(CH₂)ₚ—CH₃ group or a (CF₂)ₚ—CF₃ group, wherein p is 2, 3, 4 or 5; and
4) perfluoromorpholine derivatives of the formula IV′

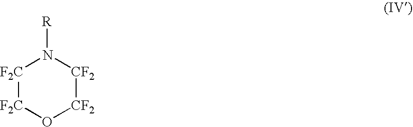

in which R is a C₁-C₄ perfluoroalkyl radical.

Suitable perfluorocycloalkyl compounds of formula I′ include, in particular, perfluoromethyl-cyclopentane and perfluoro-1,3-dimethylcyclohexane, which are sold under the names Flutec PC1® and Flutec PC3® by the company BNFL Fluorochemicals Ltd., which have respective densities of 1.26 and 1.82, as well as perfluoro-1,2-dimethylcyclobutane.

Suitable perfluoroalkane compounds of formula II′ include, in particular, dodecafluoropentane and tetradecafluorohexane, which are sold under the names PF5050® and PF 5060® by the company 3M and which have respective densities of 1.63 and 1.68, or alternatively bromo-perfluorooctyl sold under the name Foralkyl® by the company Atochem, which have density of 1.95.

Suitable fluoro compounds of formula III′ include, for example, nonafluoromethoxybutane, sold under the name MSX 4518® by the company 3M, which has a density of 1.53, and nonafluoroethoxyisobutane, which has a density of 1.43.

Finally, suitable perfluoromorpholine derivatives include, for example, 4-trifluoromethylperfluoromorpholine, sold by the company 3M under the name PF 5052®, with a density equal to 1.7.

Preferably, at least one volatile oil may be present in the composition according to the invention in an amount ranging from 10% to 99% by weight, relative to the total weight of the composition.

According to one embodiment of the present disclosure, for example, non-volatile oil may be chosen from hydrocarbon-based oils, for example of animal or plant origin, and silicone oils. As used herein, the term “hydrocarbon-based oil” means an oil mainly comprising hydrogen and carbon atoms and possibly oxygen, nitrogen, sulfur and/or phosphorus atoms.

Non-limiting examples of non-volatile hydrocarbon-based oils that may mentioned include:

a) hydrocarbon-based oils of animal origin,
b) hydrocarbon-based oils of plant origin, such as triglycerides comprising of fatty acid esters of glycerol, the fatty acids of which may have varied chain lengths from C₄ to C₂₄, these chains possibly being linear or branched, and saturated or unsaturated; these oils can be for example heptanoic or octanoic acid triglycerides, or alternatively wheat germ oil, sunflower oil, grape seed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy seed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stearineries Dubois or those sold under the names Miglyol 810®, 812® and 818® by the company Dynamit Nobel,
c) synthetic ethers comprising from 10 to 40 carbon atoms,
d) linear or branched hydrocarbons of mineral or synthetic origin, such as petroleum jelly, polydecenes, hydrogenated polyisobutene such as Parleam®, squalane and liquid paraffins, and mixtures thereof,
e) synthetic esters, for instance oils of formula R₁COOR₂ wherein R₁ is chosen from linear and branched fatty acid residues comprising from 1 to 40 carbon atoms and R₂ is chosen from a hydrocarbon-based chain, which can be, for example branched, comprising from 1 to 40 carbon atoms, for example where R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, isodecyl neopentanoate, 2-ethylhexyl palmitate, isostearyl isostearate, 2-hexyldecyl laurate, 2-octyldecyl palmitate, 2-octyldodecyl myristate, or alcohol or polyalcohol heptanoates, octanoates, decanoates or ricinoleates, for instance propylene glycol dioctanoate; hydroxylated esters, for instance isostearyl lactate, diisostearyl malate or 2-octyidodecyl lactate; polyol esters and pentaerythritol esters,
f) fatty alcohols that are liquid at room temperature with a branched and or unsaturated carbon-based chain comprising from 12 to 26 carbon atoms, for instance octyldodecanol, isostearyl alcohol, oleyl alcohol, 2-hexyldecanol, 2-butyloctanol or 2-undecylpentadecanol,
g) higher fatty acids such as oleic acid, linoleic acid or linolenic acid, and mixtures thereof.

The composition according to the present disclosure may be non-volatile silicone oils such as polydimethylsiloxanes (PDMS), polydimethylsiloxanes comprising alkyl or alkoxy groups, which are pendent and/or at the end of a silicone chain, these groups each comprising from 2 to 24 carbon atoms, phenyl silicones, for instance phenyl trimethicones, phenyl dimethicones, phenyltrimethyl-siloxydiphenylsiloxanes, diphenyl dimethicones and diphenylmethyldiphenyltrisiloxanes, and mixtures thereof.

The at least one non-volatile oil may be present in the composition according to the disclosure in an amount ranging from 1% to 60% by weight, such as ranging from 3% to 50% by weight, for example ranging from 5% to 35% by weight and further, for example, ranging from 10% to 30% by weight, relative to the total weight of the composition.

The compositions according to the present disclosure may also comprise at least one fatty substance other than the oils mentioned above, for instance waxes or pasty fatty substances.

As used herein, the term “waxes” means a fatty substance that is solid at room temperature. Among waxes that may be used according to the disclosure, non-limiting mention may be made of:

a) waxes of animal origin such as beeswax, spermaceti, lanoline wax and lanoline derivatives, plant waxes such as carnauba wax, candelilla wax, Ouricury wax, Japan wax, cocoa butter, cork fibre wax or sugarcane wax,
b) mineral waxes, for example paraffin wax, petroleum jelly wax, lignite wax, microcrystalline waxes or ozokerites,
c) synthetic waxes, among which are polyethylene waxes, polytetrafluoroethylene waxes and the waxes obtained by Fisher-Tropsch synthesis,
d) silicon waxes, for example substituted linear polysiloxanes; non-limiting examples that may be mentioned include silicon polyether waxes, alkyl or alkoxy dimethicones comprising from 16 to 45 carbon atoms, and alkylmethicones, for instance the C₃₀-C₄₅ alkyl methicone sold under the trade name “AMS C30” by Dow Corning,
e) hydrogenated oils that are solid at 25° C., such as hydrogenated castor oil, hydrogenated jojoba oil, hydrogenated palm oil, hydrogenated talo, hydrogenated coconut oil and fatty esters that are solid at 25° C., for instance the C20-C40 alkyl stearate sold under the trade name “Kester Wax K82H” by the company Koster Keunen; and mixtures thereof.

At least one wax, when present, can be present in an amount ranging from 0.05% to 5% by weight, and for example, from 0.1% to 4% by weight, relative to the total weight of the composition.

The compositions according to the present disclosure may also comprise at least one partially crosslinked elastomeric solid organopolysiloxane. The term “elastomer” means a flexible, deformable which has viscoelastic properties.

The elastomeric solid organopolysiloxane, used in this invention, can be selected from cross-linked polymers described for example in EP-A-0 295 886.

According to such an application, the elastomeric organopolysiloxanes are prepared by addition reactions and crosslinking, in the presence of a platinum-type catalyst, of at least:

(a) one organopolysiloxane containing at least two lower alkenyl groups per molecule, these alkenyl groups containing two to six carbon atoms; and
(b) one organopolysiloxane containing at least two hydrogen atoms linked to a silicon atom per molecule.

The elastomeric organopolysiloxanes used in the composition of the invention can also be selected from those described for example in U.S. Pat. No. 5,266,321. According to this patent, the elastomeric organopolysiloxanes are selected, in particular, from:

i) organopolysiloxanes comprising R₂SiO and RSiO_{1,5} units, and optionally R₃SiO_{0,5}, and/or SiO₂ in which the radicals R, independently of each other, represent hydrogen, an alkyl radical such as methyl, ethyl or propyl, an aryl radical such as phenyl or tolyl, or an unsaturated aliphatic group such as vinyl, the weight ratio of the units R₂SiO to the units RSiO_{1,5} ranging from 1/1 to 30/1;
ii) organopolysiloxanes insoluble and swelled in silicone oil, prepared by combining an hydrogenoorganopolysiloxane (1) and a organopolysiloxane (2) containing unsaturated aliphatic groups, such that the amount of hydrogen or of unsaturated aliphatic groups in (1) and (2) respectively ranges from 1 to 20 mol. % when the organopolysiloxane is non-cyclic, and from 1 to 50 mol. % when the organopolysiloxane is cyclic.

The organopolysiloxanes in the composition of the invention are, for example, those sold under the names KSG 6 from Shin Etsu, Trefil E-505C or Trefil E-506C from Dow Corning, Gransil (SR-CYC, SR DMF10, SR-DC556) from Grant Industries, or those sold in the form of preconstituted gels: KSG 15, KSG 17, KSG 16, KSG 18, KSG 26A from Shin-Etsu, Gransil SR 5CYC gel, Gransil SR DMF 10 gel, Gransil SR DC5556 gel from Grant Industries, 1229-02-167 and 1229-02-168 from General Electric, or mixtures thereof.

The composition may comprise a fatty phase formed from the oils and fatty substances described above, present in an amount ranging from 10% to 80% by weight, and for instance from 20% to 70% by weight, relative to the total weight of the composition.

The compositions according to the disclosure may comprise an oil-thickening agent chosen from polymeric thickeners and mineral thickeners. Such polymeric thickeners are described in US-A-2002/005562 and in U.S. Pat. No. 5,221,534.

In one embodiment of the present disclosure, at least one polymeric thickener may be an amorphous block copolymer of styrene and of olefin. At least one polymeric thickener may be hydrogenated to reduce the residual ethylenic unsaturations after polymerization of the monomers.

The polymeric thickener may be an optionally hydrogenated copolymer comprising styrene blocks and comprising C₃-C₄ ethylene/alkylene blocks.

At least one polymeric thickener may be present in an amount ranging from 0.1% to 10% by weight, for example ranging from 0.5% to 5% by weight and for example ranging from 1% to 3% by weight, relative to the total weight of the composition.

The composition may also comprise at least one mineral oil-thickening agent such as an organophilic clay or pyrogenic silicas.

Organophilic clays are clays modified with chemical compounds that make the clay able to swell in oily media. Clays are products that are already well known perse, which are described, for example, in the publication “Mineralogie des argiles [Mineralogy of clays], S. Caillere, S. Henin, M. Rautureau, 2nd Edition 1982, Masson,” the teaching of which is incorporated herein by way of reference.

Clays are silicates comprising a cation that may be chosen from calcium, magnesium, aluminium, sodium, potassium and lithium cations, and mixtures thereof. At least one organophilic clay may be chosen from montmorillonite, bentonite, hectorite, attapulgite and sepiolite. In one embodiment of the present disclosure, the at least one clay is a bentonite or a hectorite. These clays may be modified with at least one chemical compound chosen from quaternary amines, tertiary amines, mine acetates, imidazolines, mine soaps, fatty sulfates, alkyl aryl sulfonates and amine oxides, and mixtures thereof.

Non-limiting examples of organophilic clays that may be used include quaternium-18 bentonites such as those sold under the names Bentone 3, Bentone 38 and Bentone 38V by the company Rheox, Tixogel VP by the company United Catalyst, Claytone 34, Claytone 40 and Claytone XL by the company Southern Clay; stearalkonium bentonites such as those sold under the names Bentone 27 by the company Rheox, Tixogel LG by the company United Catalyst and Claytone AF and Claytone APA by the company Southern Clay; quaternium-18/benzalkonium bentonites such as those sold under the names Claytone HT and Claytone PS by the company Southern Clay.

The pyrogenic silicas may be obtained by high temperature hydrolysis of a volatile silicon compound in an oxhydric flame, producing a finely divided silica. This process makes it possible, for example, to obtain hydrophilic silicas having a large number of silanol groups at their surface. Such hydrophilic silicas are sold, for example, under the names Aerosil 130®, Aerosil200®, Aerosil255®, Aerosil 300® and Aerosil 380® by the company Degussa, and Cab-0-Sil HS-5®, Cab-0-Sil EH-5®, Cab-0-Sil LM-130®, Cab-0-Sil MS-55® and Cab-0-Sil M-5® by the company Cabot.

It is possible to chemically modify the surface of the said silica, via a chemical reaction generating a reduction in the number of silanol groups. It is for example possible to substitute silanol groups with hydrophobic groups: a hydrophobic silica is then obtained. The hydrophobic groups may be chosen from:

a) trimethylsiloxyl groups, which are obtained for example by treating pyrogenic silica in the presence of hexamethyldisilazane. Silicas thus treated are known as “silica silylate” according to the CTFA (6th Edition, 1995); sold, for example, under the references Aerosil R812® by the company Degussa and Cab-0-Sil TS-530® by the company Cabot;
b) dimethylsilyloxyl or polydimethylsiloxane groups, which are obtained for example by treating pyrogenic silica in the presence of polydimethylsiloxane or dimethyldichlorosilane. Silicas thus treated are known as “silica dimethyl silylate” according to the CTFA (6th Edition, 1995); sold, for example, under the references Aerosil R972® and Aerosil R974® by the company Degussa and Cab-0-Sil TS-610® and Cab-O-Sil TS-720® by the company Cabot.

At least one mineral oil-thickening agent may be present in the composition in an amount ranging from 0.5% to 7% by weight, such as in an amount ranging from 1% to 5% by weight, and for example ranging from 1% to 3% by weight, relative to the total weight of the composition.

At least one oil-thickening agent may be present in the composition in a total amount ranging from 0.1% to 10% by weight, such as ranging from 1% to 7% by weight and for instance ranging from 2% to 6% by weight, relative to the total weight of the composition.

In addition, the presence of so-called “fatty carriers” should lead to increased tanning intensity. The substances called fatty carriers according to the invention are generally also referred to as “sluices” since they transport the self-tanning agent molecules to deeper layers of the stratum corneum. Fatty carriers to be mentioned here are, in particular, ceramides, cholesterol, phospholipids, cholesteryl sulphate, cholesteryl phosphate, phosphatidylcholine, lecithin and/or empty liposomes.

According to the invention, phospholipids means the following substances: phosphatidic acids, the actual lecithins, cardolipins, lysophospholipids, lysolecithins, plasmalogens, phosphosphingolipids, sphingomyelins.

Phosphatidic acids are glycerol derivatives which are esterified in the 1^{st} and 2^{nd} position with fatty acids (1^{st} position: mostly saturated, 2^{nd} position: mostly mono- or polyunsaturated), on atom 3. In the phosphatidic acids occurring in human or animal tissue, the phosphate radical is mostly esterified with amino alcohols, such as choline (lecithin=3-sn-phosphatidylcholine) or 2-aminoethanol (ethanolamine) or L-serine (cephalin=3-sn-phosphatidylethanolamine or sn-phosphatidyl-L-serine), with myo-inositol to give the phosphoinositides common in tissues [1-(3-sn-phosphatidyl)-d-myo-inositols], with glycerol to give phosphatidyl-glycerols. Particular preference is given to lecithins. Cardiolipins (1,3-bisphosphatidylglycerols) are phospholipids comprising two phosphatidic acids joined via glycerol. Lysophospholipids are obtained when an acyl radical is cleaved off from phospholipids by phospholipase A (e.g. lysolecithins). The phospholipids also include plasmalogens, in which instead of a fatty acid in the 1 position, an aldehyde (in the form of an enol ether) is bonded; the O-1-sn-alkenyl compounds corresponding to the phosphatidylcholines are, for example, called phosphatidalcholines.

Lecithin types to be used advantageously are chosen from crude lecithins which have been deoiled and/or fractionated and/or spray-dried and/or acetylated and/or hydrolysed and/or hydrogenated.

According to the invention, use is advantageously made of ceramides, cholesterol, phospholipids, fatty acids, cholesteryl sulphate, cholesteryl phosphate, phosphatidylcholine, lecithin and/or empty liposomes.

Phospholipids to be used advantageously according to the invention can, for example, be acquired commercially under the trade names Phospholipon 25 or Phospholipon 90 (Natterman), Emulmetik 120 (Lucas Meyer), Sternpur E (Stern), Sternpur PM (Stern), Nathin 3KE (Stern), Phospholipon 90H (Nattermann/Rhone-Poulenc), Lipoid S 100 (Lipoid). According to the invention, the preferred concentration of fatty carriers is in the range from 0.1 to 20 percent by weight.

The preparations according to the invention can, moreover, comprise further customary gentle or skincare active ingredients. These may in principle be all active ingredients known to the person skilled in the art. These may be chromone derivatives. In this connection, the term chromone derivative is preferably understood as meaning certain chromen-2-one derivatives which are suitable as active ingredients for the preventative treatment of human skin and human hair against ageing processes and harmful environmental influences

A protective effect against oxidative stress or against the effect of free radicals of the formulations according to the invention can be achieved when the preparations comprise one or more antioxidants, the person skilled in the art being presented with no difficulties at all in selecting antioxidants which act suitably rapidly or in a time-delayed manner. There are many proven substances known from the specialist literature which can be used as antioxidants, e.g. amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserin), carotenoids, carotenes (e.g. [alpha]-carotene, [beta]-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, [gamma]-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulphoximine compounds (e.g. buthionine sulphoximines, homocysteine sulphoximine, buthionine sulphones, penta-, hexa-, heptathionine sulphoximine) in very low tolerated doses (e.g. pmol to [mu]mol/kg), and also (metal) chelating agents (e.g. [alpha]-hydroxy fatty acids, palmitic acid, phytic acid, lactoferrin), [alpha]-hydroxy acids (e.g. citric acid, lactic acid, malic acid), humic acid, bile acid, bile extract, bilirubin, biliverdin, EDTA, EGTA and derivatives thereof, unsaturated fatty acids and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate), and coniferyl benzoate of benzoin resin, rutinic acid and derivatives thereof, [alpha]-glycosylrutin, ferulic acid, furfurylideneglucitol, carnosine, butylhydroxytoluene, butylhydroxyanisole, nordihydroguaiaretic acid, tri-hydroxy-butyrophenone, quercitin, uric acid and derivatives thereof, mannose and derivatives thereof, zinc and derivatives thereof (e.g. ZnO, ZnSO4), selenium and derivatives thereof (e.g. selenomethionine), stilbenes and derivatives thereof (e.g. stilbene oxide, trans-stilbene oxide).

Mixtures of antioxidants are likewise suitable for use in the cosmetic preparations according to the invention. Known and commercial mixtures are, for example, mixtures comprising, as active ingredients, lecithin, L-(+)-ascorbyl palmitate and citric acid (e.g. Oxynex(R) AP), natural tocopherols, L-(+)-ascorbyl palmitate, L-(+)-ascorbic acid and citric acid (e.g. Oxynex(R) K LIQUID), tocopherol extracts from natural sources, L-(+)-ascorbyl palmitate, L-(+)-ascorbic acid and citric acid (e.g. Oxynex(R) L LIQUID), DL-[alpha]-tocopherol, L-(+)-ascorbyl palmitate, citric acid and lecithin (e.g. Oxynex(R) LM) or butylhydroxytoluene (BHT), L-(+)-ascorbyl palmitate and citric acid (e.g. Oxynex(R) 2004). Antioxidants of this type are used with compounds of the formula I in such compositions usually in ratios in the range from 1000:1 to 1:1000, preferably in amounts of from 100:1 to 1:100.

The preparations according to the invention may comprise vitamins as further ingredients. Preferably, vitamins and vitamin derivatives chosen from vitamin A, vitamin A propionate, vitamin A palmitate, vitamin A acetate, retinol, vitamin B, thiamine chloride hydrochloride (vitamin B1), riboflavin (vitamin B2), nicotinamide, vitamin C (ascorbic acid), vitamin D, ergocalciferol (vitamin D2), vitamin E, DL-[alpha]-tocopherol, tocopherol E acetate, tocopherol hydrogensuccinate, vitamin K1, esculin (vitamin P active ingredient), thiamine (vitamin B1), nicotinic acid (niacin), pyridoxine, pyridoxal, pyridoxamine, (vitamin B6), pantothenic acid, biotin, folic acid and cobalamin (vitamin B12) are present in the cosmetic preparations according to the invention, particularly preferably vitamin A palmitate, vitamin C and derivatives thereof, DL-[alpha]-tocopherol, tocopherol E acetate, nicotinic acid, pantothenic acid and biotin. Vitamins are used here with compounds of the formula I usually in ratios in the range from 1000:1 to 1:1000, preferably in amounts of from 100:1 to 1:100.

Among the phenols with an antioxidative effect, the polyphenols, some of which occur as natural substances, are particularly interesting for applications in the pharmaceutical, cosmetic or nutrition field. For example, the flavonoids or bioflavonoids, known primarily as plant dyes, often have an antioxidative potential. Effects of the substitution pattern of mono- and dihydroxyflavones are dealt with by K. Lemanska, H. Szymusiak, B. Tyrakowska, R. Zielinski, I. M. C. M. Rietjens; Current Topics in Biophysics 2000, 24(2), 101-108. It is observed therein that dihydroxyflavones with an OH group adjacent to the keto function or OH groups in 3′4′ or 6,7 or 7,8 position have antioxidative properties, whereas some other mono- and dihydroxy-flavones have no antioxidative properties.

Quercetin (cyanidanol, cyanidenolon 1522, meletin, sophoretin, ericin, 3,3′,4′,5,7-pentahydrooxyflavone) is often specified as a particularly effective antioxidant (e.g. C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology & Medicine 2001, 31(7), 869-881 investigate the pH dependency of the antioxidative effect of hydroxyflavones. Over the entire pH range, quercetin exhibits the highest activity of the investigated structures.

In addition, the preparations according to the invention can also comprise dyes and colour pigments. The dyes and colour pigments can be chosen from the corresponding positive list of the Cosmetics Directive or the EC list of cosmetic colorants. In most cases, they are identical to the dyes permitted for foods. Advantageous colour pigments are, for example, titanium dioxide, mica, iron oxides (e.g. Fe₂O₃, Fe₃O₄, FeO(OH)) and/or tin oxide. Advantageous dyes are, for example, carmine, Prussian blue, chromium oxide green, ultramarine blue and/or manganese violet.

The color additive can be used to impart a particular color (e.g., white, off-white, brown (including light brown, dark brown, tan, creme, caramel, and copper), yellow, orange, blue, green, red, etc.) to the composition. In certain non-limiting embodiments, the color additive provides the composition with a brown tint. The brown tint can resemble the color of tanned skin. When the composition is applied to the skin (application can include but is not limited to spreading on and/or rubbing the composition onto the skin), the brownish tint, for example, remains visible. The tint can remain visible even after the composition dries on the skin and until it is washed off or otherwise physically removed. An advantage of this is that it can provide the skin with an immediate tanning appearance.

It may also be favourable to choose as dye one or more substances from the following group: 2,4-dihydroxyazobenzene, 1-(2′-chloro-4′-nitro-1′-phenylazo)-2-hydroxynaphthalene, Ceres Red, 2-(4-sulpho-1-naphthylazo)-1-naphthol-4-sulphonic acid, calcium salt of 2-hydroxy-1,2′-azonaphthalene-1′-sulphonic acid, calcium and barium salts of 1-(2-sulpho-4-methyl-1-phenylazo)-2-naphthylcarboxylic acid, calcium salt of 1-(2-sulpho-1-naphthylazo)-2-hydroxynaphthalene-3-carboxylic acid, aluminium salt of 1-(4-sulpho-1-phenylazo)-2-naphthol-6-sulphonic acid, aluminium salt of 1-(4-sulpho-1-naphthylazo)-2-naphthol-3,6-disulphonic acid, 1-(4-sulpho-1-naphthylazo)-2-naphthol-6,8-disulphonic acid, aluminium salt of 4-(4-sulpho-1-phenylazo)-1-(4-sulphophenyl)-5-hydroxypyrazolone-3-carboxylic acid, aluminium and zirconium salts of 4,5-dibromofluorescein, aluminium and zirconium salts of 2,4,5,7-tetrabromofluorescein, 3′,4′,5′,6′-tetrachloro-2,4,5,7-tetrabromofluorescein and its aluminium salt, aluminium salt of 2,4,5,7-tetraiodofluorescein, aluminium salt of quinophthalone disulphonic acid, aluminium salt of indigo disulphonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extract, [beta]-carotene or cochineal.

Also advantageous for the purposes of the present invention are gel creams with a content of pearlescent pigments. Preference is given in particular to the types of pearlescent pigments listed below:

Natural pearlescent pigments, such as, for example “pearl essence” (guanine/hypoxanthin mixed crystals from fish scales) and “mother of pearl” (ground mussel shells); monocrystalline pearlescent pigments, such as, for example, bismuth oxychloride (BiOCl); layer-substrate pigments, e.g. mica/metal oxide.

Bases for pearlescent pigments are, for example, pulverulent pigments or castor oil dispersions of bismuth oxychloride and/or titanium dioxide, and bismuth oxychloride and/or titanium dioxide on mica. The lustre pigment listed under CIN 77163, for example, is particularly advantageous.

Particular preference is given to pearlescent pigments which are prepared using SiO₂. Such pigments, which may also additionally have goniochromatic effects, are available e.g. under the trade name Sicopearl Fantastico from BASF.

In addition, pigments from Engelhard/Mearl based on calcium sodium borosilicate which have been coated with titanium dioxide can advantageously be used. These are available under the name Reflecks. In addition to the colour, they have a glitter effect as a result of their particle size.

In addition, also particularly advantageous are effect pigments which are obtainable under the trade name Metasomes Standard/Glitter in various colours (yellow, red, green, blue) from Flora Tech. The glitter particles are present here in mixtures with various auxiliaries and dyes (such as, for example, the dyes with the Colour Index (CI) Numbers 19140, 77007, 77289, 77491).

The dyes and pigments may be present either individually or in a mixture, and can be mutually coated with one another, different coating thicknesses generally giving rise to different colour effects. The total amount of dyes and colour-imparting pigments is advantageously chosen from the range from e.g. 0.1% by weight to 30% by weight, preferably from 0.5 to 15% by weight, in particular from 1.0 to 10% by weight, in each case based on the total weight of the preparations.

Lipophilic compounds which may be used in combination with the compounds of formula (I) or (II) or (III) are for example:

Esters: acetyl triethylhexyl citrate, acetyl trihexyl citrate, acetylated hydrogenated castor oil, acetylated sucrose distearate, arachidyl erucate, arachidyl propionate, ascorbyl tetraisopalmitate, ascorbyl tocopheryl maleate, behenyl benzoate, benzyl laurate, benzyl ursolate, bis-(trioctyldodecyl citryl) dilinoleate, bis-behenyl/isostearyl/phytosteryl dimer dilinoleyl dimer dilinoleate, bis-behenyl/phytosteryl dimer dilinoleate, bis-isostearyl dimer dilinoleyl dimer dilinoleate, butoxyethyl stearate, butyl acetyl ricinoleate, butyl babassuate, butyl isostearate, butyl myristate, butyl oleate, butyl stearate, butylene glycol behenate, butylene glycol laurate, butylene glycol myristate, butylene glycol oleate, butylene glycol palmitate, butylene glycol stearate, butyloctyl benzoate, C₁₀-C₄₀ isoalkyl acid phytosterol esters, C₁₁-C₁₅ pareth-12 stearate, C₁₁-C₁₅ pareth-3 oleate, C₁₁-C₁₅ pareth-3 stearate, C₁₂-C₁₃ alkyl ethylhexanoate, C₁₂-C₁₃ alkyl lactate, C₁₂-C₁₅ alkyl benzoate, C₁₂-C₁₅ alkyl ethylhexanoate, C₁₂-C₁₅ alkyl lactate, C₁₂-C₁₅ pareth-12 oleate, C₁₂-C₁₅ pareth-3 benzoate, C₁₂-C₁₅ pareth-9 hydrogenated tallowate, C₁₄-C₁₅ dialkyl carbonate, C₁₄-C₁₆ glycol palmitate, C₁₄-C₁₈ acid glycol ester, C₁₄-C₁₈ alkyl ethylhexanoate, C₁₆-C₁₇ alkyl benzoate, C₁₈-C₂₀ glycol isostearate, C₁₈-C₂₈ alkyl acetate, C₁₈-C₃₀ acid glycol ester, C₁₈-C₃₆ acid glycol ester, C₂₀-C₃₀ glycol isostearate, C₂₃-C₄₃ acid pentaerythritol tetraester, C₃₂-C₃₆ isoalkyl stearate, castor oil benzoate, ceteareth-6 olivate, cetearyl ethylhexanoate, cetearyl isononanoate, cetearyl palmitate, cetyl acetate, cetyl babassuate, cetyl C₁₂-C₁₅ pareth-8 carboxylate, cetyl C₁₂-C₁₅-pareth-9 carboxylate, cetyl caprate, cetyl caprylate, cetyl dimethyloctanoate, cetyl ethylhexanoate, cetyl glycol isostearate, cetyl isononanoate, cetyl combo buterate, cetyl lactate, cetyl laurate, cetyl oleate, cetyl PPG-2 isodeceth-7 carboxylate, chimyl isostearate, chimyl stearate, cholesteryl succinate, coco-caprylate/caprate, coco-rapeseedate, cycloartenyl ferulate, decyl castorate, decyl hempsee-date, decyl isostearate, decyl jojobate, decyl oleate, decyl palmitate, decyl succinate, dibutyl adipate, dibutyl sebacate, dibutyldecyl IPDI, dibutyloctyl IPDI, dibutyloctyl malate, dibutyloctyl sebacate, di-C₁₂-C₁₃ alkyl malate, di-C₁₂-C₁₃ alkyl tartrate, di-C₁₂-C₁₅ alkyl adipate, di-C₁₂-C₁₅ alkyl fumarate, di-C₁₂-C₁₅ alkyl maleate, di-C₁₄-C₁₅ alkyl tartrate, dicaprylyl carbonate, dicaprylyl maleate, dicetyl adipate, dicocoyl pentaerythrityl distearyl citrate, diethyl adipate, diethyl palmitoyl aspartate, diethyl sebacate, diethylene glycol diethylhexanoate/diisononanoate, diethylene glycol diisononanoate, diethylhexyl 2,6-naphthalate, diethylhexyl adipate, diethylhexyl carbonate, diethylhexyl fumarate, diethylhexyl malate, diethylhexyl maleate, diethylhexyl succinate, diglyceryl sebacate/isopalmitate, diglyceryl stearate malate, diheptylundecyl adipate, dihexyl adipate, dihexyldecyl adipate, dihexyldecyl IPDI, dihexyldecyl lauroyl glutamate, dihexyldecyl sebacate, dihydrocholesteryl butyrate, dihydrocholesteryl isostearate, dihydrocholesteryl macadamiate, dihydrocholesteryl nonanoate, dihydrocholesteryl octyldecanoate, dihydrocholesteryl oleate, diisoamyl malate, diisobutyl adipate, diisocetyl adipate, diisocetyl dodecanedioate, diisodecyl adipate, diisononyl adipate, diisooctyl adipate, diisopropyl adipate, diisopropyl dimer dilinoleate, diisopropyl sebacate, diisostearyl adipate, diisostearyl fumarate, diisostearyl glutarate, diisostearyl malate, diisostearyl sebacate, dilauryl citrate, dimer dilinoleyl diisostearate, dimer dilinoleyl dimer dilinoleate, dimer dilinoleyl isostearate, dimethicone PEG-7 avocadoate, dimethicone PEG-7 cocoate, dimethicone PEG-7 isostearate, dimethicone PEG-7 octyldodecyl citrate, dimethicone PEG-7 olivate, dimethicone PEG-7 phthalate, dimethicone PEG-8 adipate, dimethicone PEG-8 beeswax, dimethicone PEG-8 borageate, dimethicone PEG-8 meadowfoamate, dimethiconol borageate, dimethiconol dhupa butterate, dimethiconol Illipe butterate, dimethiconol kokum butterate, dimethiconol lactate, dimethiconol meadowfoamate, dimethiconol mohwa butterate, dimethiconol sal butterate, dimethyl adipate, dimethyl succinate, dimyristyl tartrate, dioctadecanyl didecyltetradecanoate, dioctadecanyl ditetradecyloctadecanoate, dioctyldodeceth-2 lauroyl glutamate, dioctyldodeceth-5 lauroyl glutamate, dioctyldodecyl adipate, dioctyldodecyl lauroyl glutamate, dioctyldodecyl sebacate, dipentaerythrityl hexahydroxystearate/hexastearate/-hexahydrogenated rosinate, dipentaerythrityl pentaisononanoate, dipentaerythrityl pentaisostearate, di-PPG-2 myreth-10 adipate, di-PPG-3 ceteth-4 adipate, di-PPG-3 myristyl ether adipate, dipropyl adipate, dipropylene glycol caprylate, dipropylene glycol dibenzoate, dipropylene glycol salicylate, disteareth-2 lauroyl glutamate, disteareth-5 lauroyl glutamate, distearyl citrate, ditridecyl adipate, ditrimethylolpropane isostearate/sebacate, ditrimethylolpropane myristate/stearate, ditrimethylolpropane triethylhexanoate, erythrityl triethylhexanoate, ethoxydiglycol behenate, ethoxydiglycol isostearate, ethoxydiglycol oleate, ethyl almondate, ethyl apricot kernelate, ethyl arachidonate, ethyl avocadate, ethyl caprate, ethyl isostearate, ethyl jojobate, ethyl laurate, ethyl linoleate, ethyl linolenate, ethyl macadamiate, ethyl minkate, ethyl morrhuate, ethyl myristate, ethyl oleate, ethyl olivate, ethyl palmitate, ethyl pelargonate, ethyl persate, ethyl ricinoleate, ethyl stearate, ethyl turtlate, ethyl undecylenate, ethyl wheat germate, ethylhexyl acetoxystearate, ethylhexyl adipate/palmitate/stearate, ethylhexyl benzoate, ethylhexyl benzyldioxopiperazyl acetate, ethylhexyl caprylate/caprate, ethylhexyl cocoate, ethylhexyl ethylhexanoate, ethylhexyl hydroxystearate, ethylhexyl hydroxystearate benzoate, ethylhexyl hydroxystearoyl hydroxystearate, ethylhexyl isononanoate, ethylhexyl isopalmitate, ethylhexyl isostearate, ethylhexyl laurate, ethylhexyl linoleoyl stearate, ethylhexyl myristate, ethylhexyl neopentanoate, ethylhexyl oleate, ethylhexyl palmitate, ethylhexyl pelargonate, ethylhexyl stearate, glycereth-5 lactate, glycereth-7 benzoate, glycereth-7 diisononanoate, glycereth-7 lactate, glyceryl stearate dimer dilinoleate copolymer, glyceryl triacetyl hydroxystearate, glyceryl triacetyl ricinoleate, glycol cetearate, glycol dipalmate/palm kernelate/olivate/macadamiate, glycol dipivalate, glycol ethylhexanoate, glycol hydroxystearate, glycol montanate, glycol oleate, glycol palmitate, glycol ricinoleate, glycol stearate, glycol tallate, glycol/butylene glycol montanate, heptylundecyl hydroxystearate, hexanediol beeswax, hexanediol disalicylate, hexanetriol beeswax, hexyl isostearate, hexyl laurate, hexyldecyl benzoate, hexyldecyl ethylhexanoate, hexyldecyl hexyldecanoate, hexyldecyl laurate, hexyldecyl myristoyl methylaminopropionate, hexyldecyl stearate, hydrogenated ethylhexyl olivate, hydrogenated ethylhexyl sesamate, hydroxycetyl isostearate, hydroxypropyl dimethicone behenate, isoamyl laurate, isobutyl myristate, isobutyl palmitate, isobutyl pelargonate, isobutyl stearate, isobutyl tallowate, isocetyl ethylhexanoate, isocetyl isodecanoate, isocetyl isostearate, isocetyl laurate, isocetyl linoleoyl stearate, isocetyl palmitate, isocetyl stearate, isodeceth-2 cocoate, isodecyl citrate, isodecyl cocoate, isodecyl ethylhexanoate, isodecyl hydroxystearate, isodecyl isononanoate, isodecyl laurate, isodecyl myristate, isodecyl neopentanoate, isodecyl oleate, isodecyl palmitate, isodecyl stearate, isohexyl caprate, isohexyl laurate, isohexyl neopentanoate, isohexyl palmitate, isomerized jojoba oil, isononyl ferulate, isononyl isononanoate, isopropyl arachidate, isopropyl avocadate, isopropyl babassuate, isopropyl behenate, isopropyl C₁₂-C₁₅-pareth-9 carboxylate, isopropyl hydroxystearate, isopropyl isostearate, isopropyl jojobate, isopropyl laurate, isopropyl linoleate, isopropyl myristate, isopropyl oleate, isopropyl palmitate, isopropyl PPG-2 isodeceth-7 carboxylate, isopropyl ricinoleate, isopropyl stearate, isopropyl tallowate, isosorbide dicaprylate, isosorbide laurate, isosteareth-10 stearate, isostearyl acetate, isostearyl acetyl glutaminate, isostearyl avocadate, isostearyl benzoate, isostearyl ethylhexanoate, isostearyl ferulate, isostearyl glycolate, isostearyl hydroxystearate, isostearyl isononanoate, isostearyl isostearate, isostearyl lactate, isostearyl laurate, isostearyl linoleate, isostearyl myristate, isostearyl neopentanoate, isostearyl palmitate, isotridecyl isononanoate, isotridecyl stearate, kojic dipalmitate, kojic isopalmitate, laneth-10 acetate, laneth-9 acetate, laureth-2 acetate, laureth-2 benzoate, laureth-2 ethylhexanoate, lauryl cocoate, lauryl ethylhexanoate, lauryl isostearate, lauryl lactate, laurylgluconamide palmitates, lignoceryl erucate, linoleyl lactate, meadowfoam estolide, methyl acetyl ricinoleate, methyl behenate, methyl benzoate, methyl caproate, methyl caprylate, methyl caprylate/caprate, methyl cocoate, methyl dehydroabietate, methyl gluceth-20 benzoate, methyl glucose caprylate/caprate, methyl glucose dioleate, methyl glucose isostearate, methyl glucose laurate, methyl glucose sesquicaprylate/sesquicaprate, methyl glucose sesquicocoate, methyl glucose sesquiisostearate, methyl glucose sesquilaurate, methyl glucose sesquioleate, methyl glucose sesquistearate, methyl hydrogenated rosinate, methyl hydroxystearate, methyl isostearate, methyl laurate, methyl linoleate, methyl myristate, methyl oleate, methyl palmate, methyl palmitate, methyl pelargonate, methyl ricinoleate, methyl rosinate, methyl soyate, methyl stearate, methyl sunflowerseedate, monoglycerides acetate, monoglycerides citrate, monoglycerides lactate, monoglycerides succinate, monoglycerides/diacetyl tartarate esters, myreth-2 myristate, myreth-3 caprate, myreth-3 ethylhexanoate, myreth-31Laurate, myreth-3 myristate, myreth-3 palmitate, myristyl acetate, myristyl alaninate, myristyl ethylhexanoate, myristyl isostearate, myristyl lactate, myristyl lignocerate, myristyl neopentanoate, myristyl propionate, neopentyl glycol dicaprate, neopentyl glycol dicaprylate/dicaprate, neopentyl glycol dicaprylate/dipelargonate/dicaprate, neopentyl glycol diethylhexanoate, neopentyl glycol diheptanoate, neopentyl glycol dilaurate, nonyl acetate, octyldodecyl beeswax, octyldodecyl benzoate, octyldodecyl ethylhexanoate, octyldodecyl lactate, octyldodecyl neodecanoate, octyldodecyl neopentanoate, octyldodecyl PCA, octyldodecyl/PPG-3 myristyl ether dimer dilinoleate, oleth-2 benzoate, oleuropein aglycone, oleyl acetate, oleyl lactate, oleyl oleate, palmitoyl inulin, PEG-20 methyl glucose distearate, PEG-20 methyl glucose sesquicaprylate/sesquicaprate, PEG-20 methyl glucose sesquilaurate, PEG-3 butylene glycol laurate, PEG-3 trimethylolpropane tristearate, PEG-4 trimethylolpropane distearate, PEG-5 tricapryl citrate, PEG-5 tricetyl citrate, PEG-5 trilauryl citrate, PEG-5 trimethylolpropane trimyristate, PEG-5 trimyristyl citrate, PEG-5 tristearyl citrate, PEG-6 butylene glycol laurate, PEG-6 propylene glycol caprylate/caprate, PEG-8 propylene glycol cocoate, PEG-80 methyl glucose laurate, pentaerythrityl dioleate, pentaerythrityl distearate, pentaerythrityl rosinate, pentaerythrityl stearate, pentaerythrityl tetrabehenate/-benzoate/ethylhexanoate, pentaerythrityl tetrabenzoate, pentaerythrityl tetraethyl hexanoate/-benzoate, pentaerythrityl tetraethylhexanoate/tetramethoxycinnamate, pentaerythrityl trioleate, phenethyl benzoate, phytosteryl hydroxystearate, phytosteryl isostearyl dimer dilinoleate, phytosteryl rice branate, poloxamer 182 dibenzoate, polyperfluoroethoxymethoxy difluoroethyl PEG diisostearate, PPG-15 stearyl ether benzoate, PPG-2 isoceteth-20 acetate, PPG-20 methyl glucose ether distearate, PPG-3 dipivalate, PPG-3 myristyl ether neoheptanoate, propyl C₁₂-C₁₅ pareth-8 carboxylate, propylene glycol behenate, propylene glycol caprylate, propylene glycol ceteth-3 acetate, propylene glycol ceteth-3 propionate, propylene glycol cocoate, propylene glycol dibenzoate, propylene glycol heptanoate, propylene glycol hydroxystearate, propylene glycol isoceteth-3 acetate, propylene glycol isostearate, propylene glycol laurate, propylene glycol linoleate, propylene glycol linolenate, propylene glycol myristate, propylene glycol myristyl ether acetate, propylene glycol oleate, propylene glycol ricinoleate, propylene glycol soyate, propylene glycol stearate, quercetin caprylate, raffinose myristate, raffinose oleate, retinyl formyl aspartamate, salicylyl beeswax, starch tallowate, steareth-9 stearate, stearyl acetate, stearyl acetyl glutaminate, stearyl benzoate, stearyl citrate, stearyl glycol isostearate, stearyl lactate, stearyl/PPG-3 myristyl ether dimer dilinoleate, sucrose acetate/stearate, sucrose cocoate, sucrose dilaurate, sucrose distearate, sucrose hexaerucate, sucrose laurate, sucrose mortierellate, sucrose myristate, sucrose oleate, sucrose palmitate, sucrose pentaerucate, sucrose polybehenate, sucrose polycotton-seedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose tetrastearate triacetate, sucrose tribehenate, sucrose tristearate, tetradecylpropionates, tetrahydrofurfuryl ricinoleate, tocopheryl retinoate, tribenzoyl triricinolein, tri-C₁₂-C₁₃ alkyl citrate, tri-C₁₄-C₁₅ alkyl citrate, tricaprylyl/capryl trimellitate, tridecyl ethylhexanoate, tridecyl isononanoate, tridecyl neopentanoate, tridecyl stearate, triethylhexyl citrate, triethylhexyl trimellitate, triisodecyl trimellitate, triisopropyl citrate, trioleyl citrate.
Glyceryl ester and derivatives: Acetyl Glyceryl Ricinoleate, Acetylated Lard Glyceride, Acetylated Palm Kernel Glycerides, Argan Oil PEG-8 Esters, Bis-Diglyceryl Polyacyladipate-1, Bis-Diglyceryl Polyacyladipate-2, C10-40 Isoalkyl Acid Glyceride, C10-40 Isoalkyl Acid Triglyceride, Canola Oil Glyceride, Caprylic/Capric Glycerides, Caprylic/Capric/Coco Glycerides, Caprylic/Capric/Succinic Triglyceride, Cocoglycerides, Cottonseed Glyceride, DATEM, Di-Glyceryl Stearate Dimer Dilinoeate, Diglyceryl Stearate Malate, Diisostearoyl Polyglyceryl-3 Dimer Dilinoleate, Ethylhexyloxyglyceryl Palmitate, Fish Glycerides, Glycereth-18 Benzoate, Glycereth-20 Benzoate, Glycereth-6 C10-18 Acidate, Glycereth-7 Triacetate, Glycerin/Phthalic Acid Copolymer Hydroxypropyl C9-11 Isoalkyl Acidate, Glyceryl Abietate, Glyceryl Abietate/Maleate, Glyceryl Adipate, Glyceryl Alginate, Glyceryl Arachidate, Glyceryl Arachidonate, Glyceryl Behenate, Glyceryl Behenate/Eicosadioate, Glyceryl Caprate, Glyceryl Caprylate, Glyceryl Caprylate/Caprate, Glyceryl Citrate/Lactate/Linoleate/Oleate, Glyceryl Cocoate, Glyceryl Cocoate/Citrate/Lactate, Glyceryl Collagenate, Glyceryl Diarachidate, Glyceryl Dibehenate, Glyceryl Dibehenate/Hydrogenated Rosinate, Glyceryl Dierucate, Glyceryl Di-Hydroxystearate, Glyceryl Diisopalmitate, Glyceryl Diisostearate, Glyceryl Dilaurate, Glyceryl Dilinoleate, Glyceryl Dimyristate, Glyceryl Dimyristate/Hydrogenated Rosinate, Glyceryl Dioleate, Glyceryl Dipalmitate, Glyceryl Dipalmitoleate, Glyceryl Diricinoleate, Glyceryl Distearate, Glyceryl Erucate, Glyceryl Heptanoate, Glyceryl Hydrogenated Rosinate, Glyceryl Hydrogenated Soyate, Glyceryl Hydroxystearate, Glyceryl Isopalmitate, Glyceryl Isostearate, Glyceryl Isostearate/Myristate, Glyceryl Isostearates, Glyceryl Isotridecanoate/Stearate/Adipate, Glyceryl Lanolate, Glyceryl Laurate, Glyceryl Laurate/Oleate, Glyceryl Linoleate, Glyceryl Linolenate, Glyceryl Montanate, Glyceryl Myristate, Glyceryl Oleate, Glyceryl Oleate/Elaidate, Glyceryl Olivate, Glyceryl Palmitate, Glyceryl Palmitate Lactate, Glyceryl Palmitate/Stearate, Glyceryl Palmitoleate, Glyceryl Ricinoleate, Glyceryl Ricinoleate SE, Glyceryl Rosinate, Glyceryl Sesquioleate, Glyceryl Stearate, Glyceryl Stearate Citrate, Glyceryl Stearate Lactate, Glyceryl Stearate Succinate, Glyceryl Stearate/Acetate, Glyceryl Stearate/Malate, Glyceryl Stearates, Glyceryl Tallowate, Glyceryl Tripalmate/Palm Kernelate/Olivate/Macadamiate/-Rapeseedate, Glyceryl Undecylenate, Glyceryl/Sorbitol Oleate/Hydroxystearate, Hydrogenated Castor Oil/Sebacic Acid Copolymer, Hydrogenated Coco-Glycerides, Hydrogenated Lard Glyceride, Hydrogenated Lard Glycerides, Hydrogenated Palm Glycerides, Hydrogenated Palm Glycerides Citrate, Hydrogenated Palm Kernel Glycerides, Hydrogenated Soy Glycerides, Hydrogenated Tallow Glyceride Citrate, Hydrogenated Tallow Glyceride Lactate, Hydrogenated Tallow Glycerides, Hydrogenated Tallow Glycerides Citrate, Hydrogenated Vegetable Glycerides, Hydrogenated Vegetable Glycerides Citrate, Hydroxylated Milk Glycerides, Hydroxystearic/Linolenic/Oleic Polyglycerides, Isostearic/Myristic Glycerides, Lard Glyceride, Lard Glycerides, Lauryl Polyglyceryl-6 Cetearyl Glycol Ether, Methylsilanol PEG-7 Glyceryl Cocoate, Methylsilanol Tri-PEG-8 Glyceryl Cocoate, Oleic/Linoleic/Linolenic Polyglycerides, Palm Glycerides, Palm Kernel Glycerides, PEG-10 Castor Oil, PEG-10 Glyceryl Triisostearate, PEG-10 Glyceryl Trioleate, PEG-10 Glyceryl Tristearate, PEG-10 Hydrogenated Castor Oil, PEG-10 Olive Glycerides, PEG-10 Sunflower Glycerides, PEG-11 Avocado Glycerides, PEG-11 Babassu Glycerides, PEG-11 Castor Oil, PEG-11 Cocoa Butter Glycerides, PEG-12 Glyceryl Dioleate, PEG-12 Glyceryl Distearate, PEG-12 Palm Kernel Glycerides, PEG-13 Mink Glycerides PEG-13 Sunflower Glycerides, PEG-14 Avocado Glycerides, PEG-15 Castor Oil, PEG-15 Glyceryl Laurate, PEG-15 Glyceryl Triisostearate, PEG-15 Glyceryl Trioleate, PEG-16 Castor Oil, PEG-16 Hydrogenated Castor Oil, PEG-16 Macadamia Glycerides, PEG-18 Glyceryl Oleate/Cocoate, PEG-18 Palm Glycerides, PEG-192 Apricot Kernel Glycerides, PEG-2 Castor Oil, PEG-2 Hydrogenated Castor Oil, PEG-2 Olive Glycerides, PEG-2 Sunflower Glycerides, PEG-20 Almond Glycerides, PEG-20 Corn Glycerides, PEG-20 Evening Primrose Glycerides, PEG-20 Glyceryl Stearate, PEG-20 Glyceryl Triisostearate, PEG-20 Glyceryl Tristearate, PEG-20 Hydrogenated Palm Glycerides, PEG-25 Glyceryl Trioleate, PEG-25 Moringa Glycerides, PEG-3 Castor Oil, PEG-3 Glyceryl Cocoate, PEG-3 Glyceryl Triisostearate, PEG-3 Glyceryl Trioleate, PEG-3 Glyceryl Tristearate, PEG-30 Glyceryl Triisostearate, PEG-35 Almond Glycerides, PEG-4 Caprylic/Capric Glycerides, PEG-4 Castor Oil, PEG-4 Glyceryl Distearate, PEG-4 Glyceryl Tristearate, PEG-40 Glyceryl Triisostearate, PEG-40 Olive Glycerides, PEG-42 Babassu Glycerides, PEG-42 Mushroom Glycerides, PEG-45 Palm Kernel Glycerides, PEG-45 Safflower Glycerides, PEG-5 Castor Oil, PEG-5 Glyceryl Triisostearate, PEG-5 Glyceryl Trioleate, PEG-5 Glyceryl Tristearate, PEG-5 Hydrogenated Castor Oil, PEG-5 Hydrogenated Corn Glycerides, PEG-50 Glyceryl Triisostearate, PEG-6 Almond Glycerides, PEG-6 Caprylic/Capric Glycerides, PEG-6 Glyceryl Caprate, PEG-6 Glyceryl Tristearate, PEG-6 Hydrogenated Castor Oil, PEG-6 Hydrogenated Palm/Palm Kernel Glyceride, PEG-6 Olive Glycerides, PEG-60 Almond Glycerides, PEG-60 Corn Glycerides, PEG-60 Glyceryl Triisostearate, PEG-60 Shea Butter Glycerides, PEG-65 Hydrogenated Castor Oil, PEG-7 Caprylic/Capric Glycerides, PEG-7 Glyceryl Cocoate, PEG-7 Glyceryl Laurate, PEG-7 Glyceryl Soyate, PEG-7 Hydrogenated Castor Oil, PEG-7 Olive Glycerides, PEG-7 Sunflower Glycerides, PEG-70 Mango Glycerides, PEG-75 Cocoa Butter Glycerides, PEG-75 Meadowfoam Oil, PEG-75 Shea Butter Glycerides, PEG-75 Shorea Butter Glycerides, PEG-75 Soy Glycerides, PEG-8 Caprylic/Capric Glycerides, PEG-8 Castor Oil, PEG-8 Glyceryl Laurate, PEG-8 Hydrogenated Castor Oil, PEG-8 Hydrogenated Fish Glycerides, PEG-9 Castor Oil, PEG-9 Cocoglycerides, Polyglyceryl-10 Caprate, Polyglyceryl-10 Decahydroxystearate, Polyglyceryl-10 Decaisostearate, Polyglyceryl-10 Decalinoleate, Polyglyceryl-10 Decamacadamiate, Polyglyceryl-10 Decaoleate, Polyglyceryl-10 Decastearate, Polyglyceryl-10 Didecanoate, Polyglyceryl-10 Diisostearate, Polyglyceryl-10 Dioleate, Polyglyceryl-10 Dipalmitate, Polyglyceryl-10 Distearate, Polyglyceryl-10 Dodecacaprate, Polyglyceryl-10 Dodecacaprylate, Polyglyceryl-10 Heptaoleate, Polyglyceryl-10 Heptastearate, Polyglyceryl-10 Hexaoleate, Polyglyceryl-10 Hydroxystearate/Stearate/Eicosadioate, Polyglyceryl-10 Isostearate, Polyglyceryl-10 Mono/Dioleate, Polyglyceryl-10 Nonaerucate, Polyglyceryl-10 Nonaisostearate, Polyglyceryl-10 Palmitate, Polyglyceryl-10 Pentahydroxystearate, Polyglyceryl-10 Pentaisostearate, Polyglyceryl-10 Pentalaurate, Polyglyceryl-10 Pentalinoleate, Polyglyceryl-10 Pentaoleate, Polyglyceryl-10 Pentaricinoleate, Polyglyceryl-10 Pentastearate, Polyglyceryl-10 Tetraoleate, Polyglyceryl-10 Tridecanoate, Polyglyceryl-10 Tristearate, Polyglyceryl-2 Caprate, Polyglyceryl-2 Caprylate, Polyglyceryl-2 Diisostearate, Polyglyceryl-2 Dioleate, Polyglyceryl-2 Isopalmitate, Polyglyceryl-2 Isostearate, Polyglyceryl-2 Laurate, Polyglyceryl-2 Myristate, Polyglyceryl-2 Oleate, Polyglyceryl-2 Palmitate, Polyglyceryl-2 Sesquicaprylate, Polyglyceryl-2 Sesquiisostearate, Polyglyceryl-2 Sesquioleate, Polyglyceryl-2 Sesquistearate, Polyglyceryl-2 Stearate, Polyglyceryl-2 Tetraisostearate, Polyglyceryl-2 Tetrastearate, Polyglyceryl-2 Triisostearate, Polyglyceryl-3 Caprate, Polyglyceryl-3 Dicaprate, Polyglyceryl-3 Dicocoate, Polyglyceryl-3 Di-Hydroxystearate, Polyglyceryl-3 Diisostearate, Polyglyceryl-3 Dioleate, Polyglyceryl-3 Distearate, Polyglyceryl-3 Isostearate, Polyglyceryl-3 Laurate, Polyglyceryl-3 Methylglucose Distearate, Polyglyceryl-3 Myristate, Polyglyceryl-3 Oleate, Polyglyceryl-3 Palmitate, Polyglyceryl-3 Pentaricinoleate, Polyglyceryl-3 Ricinoleate, Polyglyceryl-3 Stearate, Polyglyceryl-3 Triisostearate, Polyglyceryl-4 Caprate, Polyglyceryl-4 Dilaurate, Polyglyceryl-4 Isostearate, Polyglyceryl-4 Laurate, Polyglyceryl-4 Oleate, Polyglyceryl-4 Pentaoleate, Polyglyceryl-4 Pentastearate, Polyglyceryl-4 Tristearate, Polyglyceryl-5 Caprate, Polyglyceryl-5 Hexastearate, Polyglyceryl-5 Isostearate, Polyglyceryl-5 Laurate, Polyglyceryl-5 Myristate, Polyglyceryl-5 Oleate, Polyglyceryl-5 Pentamyristate, Polyglyceryl-5 Tribehenate, Polyglyceryl-5 Trimyristate, Polyglyceryl-5 Trioleate, Polyglyceryl-6 Caprylate, Polyglyceryl-6 Dicaprate, Polyglyceryl-6 Diisostearate, Polyglyceryl-6 Dioleate, Polyglyceryl-6 Dipalmitate, Polyglyceryl-6 Distearate, Polyglyceryl-6 Hexaoleate, Polyglyceryl-6 Hexastearate, Polyglyceryl-6 Isostearate, Polyglyceryl-6 Laurate, Polyglyceryl-6 Myristate, Polyglyceryl-6 Octastearate, Polyglyceryl-6 Oleate, Polyglyceryl-6 Palmitate, Polyglyceryl-6 Pentaoleate, Polyglyceryl-6 Pentaricinoleate, Polyglyceryl-6 Pentastearate, Polyglyceryl-6 Polyricinoleate, Polyglyceryl-6 Stearate, Polyglyceryl-6 Tetrabehenate, Polyglyceryl-6 Tetraoleate, Polyglyceryl-8 Decaerucate/Decaisostearate/Decaricinoleate, PPG-3 Hydrogenated Castor Oil, PPG-5 Lanolin Wax Glyceride, PPG-67 Glyceryl Ether, PPG-70 Glyceryl Ether, Rapeseed Glyceride, Rapeseed Glycerides, Rubus Occidentalis Seed Oil, Sunflower Seed Oil Glyceride, Sunflower Seed Oil Glycerides, Tall Oil Glycerides, Tallow Glycerides, Tribenzoyl Triricinolein, Triisostearoyl Polyglyceryl-3 Dimer Dilinoleate, Vegetable Glycerides Phosphate, Wheat Germ Glycerides.
Alkoxylated Alcohols Esters: C12-15 Pareth-3 Benzoate, Cetyl C12-15 Pareth-8 Carboxylate, Di-PPG-2 Myreth-10 Adipate, Di-PPG-3 Ceteth-4 Adipate, Di-PPG-3 Myristyl Ether Adipate, Glycereth-5 Lactate, Glycereth-7 Benzoate, Glycereth-7 Diisononanoate, Glycereth-7 Lactate, Oleth-2 Benzoate, PEG-3 Butylene Glycol Laurate, PEG-5 Trilauryl Citrate, PEG-5 Trimyristyl Citrate, PEG-5 Tristearyl Citrate, PEG-6 Butylene Glycol Laurate, PEG-6 Propylene Glycol Caprylate/Caprate, PEG-8 Propylene Glycol Cocoate, PPG-15 Stearyl Ether Benzoate, PPG-20 Methyl Glucose Ether Distearate, PPG-3 Myristyl Ether Neoheptanoate, Methyl Gluceth-20 Benzoate.
Alkoxylated Alcohols Glyceryl Esters and Derivatives: Glycereth-18 Benzoate, Glycereth-20 Benzoate, Glycereth-7 Triacetate, PEG-10 Castor Oil, PEG-10 Hydrogenated Castor Oil, PEG-10 Olive Glycerides, PEG-10 Sunflower Glycerides, PEG-11 Avocado Glycerides, PEG-11 Babassu Glycerides, PEG-11 Castor Oil, PEG-11 Cocoa Butter Glycerides, PEG-12 Glyceryl Dioleate, PEG-12 Glyceryl Distearate, PEG-12 Palm Kernel Glycerides, PEG-13 Mink Glycerides, PEG-13 Sunflower Glycerides, PEG-14 Avocado Glycerides, PEG-15 Castor Oil, PEG-15 Glyceryl Laurate, PEG-16 Castor Oil, PEG-16 Hydrogenated Castor Oil, PEG-16 Macadamia Glycerides, PEG-18 Glyceryl Oleate/Cocoate, PEG-18 Palm Glycerides, PEG-192 Apricot Kernel Glycerides, PEG-2 Castor Oil, PEG-2 Hydrogenated Castor Oil, PEG-2 Olive Glycerides, PEG-2 Sunflower Glycerides, PEG-20 Almond Glycerides, PEG-20 Corn Glycerides, PEG-20 Evening Primrose Glycerides, PEG-20 Glyceryl Stearate, PEG-20 Hydrogenated Palm Glycerides, PEG-25 Moringa Glycerides, PEG-3 Castor Oil, PEG-3 Glyceryl Cocoate, PEG-4 Castor Oil, PEG-4 Glyceryl Distearate, PEG-40 Olive Glycerides, PEG-42 Babassu Glycerides, PEG-42 Mushroom Glycerides, PEG-45 Palm Kernel Glycerides, PEG-45 Safflower Glycerides, PEG-5 Castor Oil, PEG-5 Hydrogenated Castor Oil, PEG-5 Hydrogenated Corn Glycerides, PEG-50 Glyceryl Triisostearate, PEG-6 Almond Glycerides, PEG-6 Glyceryl Caprate, PEG-60 Almond Glycerides, PEG-60 Corn Glycerides, PEG-60 Shea Butter Glycerides, PEG-65 Hydrogenated Castor Oil, PEG-7 Glyceryl Cocoate, PEG-7 Glyceryl Laurate, PEG-7 Glyceryl Soyate, PEG-7 Hydrogenated Castor Oil, PEG-7 Olive Glycerides, PEG-7 Sunflower Glycerides, PEG-70 Mango Glycerides, PEG-75 Cocoa Butter Glycerides, PEG-75 Meadowfoam Oil, PEG-75 Shea Butter Glycerides, PEG-75 Shorea Butter Glycerides, PEG-75 Soy Glycerides, PEG-8 Castor Oil, PEG-8 Glyceryl Laurate, PEG-8 Hydrogenated Castor Oil, PEG-8 Hydrogenated Fish Glycerides, PEG-9 Castor Oil, PEG-9 Cocoglycerides, PPG-3 Hydrogenated Castor Oil, PPG-5 Lanolin Wax Glyceride.
Alkoxylated Alcohol Ethers: PPG-9 Diglyceryl Ether Glycereth-7 Trimethyl Ether, PPG-10 Cetyl Ether, PPG-10 Oleyl Ether, PPG-11 Stearyl Ether, PPG-14 Butyl Ether, PPG-15 Stearyl Ether, PPG-20 Cetyl Ether, PPG-20 Oleyl Ether, PPG-23 Oleyl Ether, PPG-28 Cetyl Ether, PPG-3 Benzyl Ether Myristate, PPG-3 Myristyl Ether, PPG-30 Cetyl Ether, PPG-30 Isocetyl Ether, PPG-30 Oleyl Ether, PPG-37 Oleyl Ether, PPG-3-Isodeceth-1, PPG-4 Lauryl Ether, PPG-4 Myristyl Ether, PPG-5 Pentaerythrityl Ether, PPG-50 Cetyl Ether, PPG-50 Oleyl Ether, PPG-65-PEG-5 Pentaerythrityl Ether, PPG-9-Steareth-3.
Fats and Oils: Argania Spinosa Kernel Oil, Argemone Mexicana Oil, Astrocaryum Vulgare Fruit Oil, Astrocaryum Vulgare Kernel Oil, Avena Sativa (Oat) Kernel Oil, Balanites Roxburghii Seed Oil, Bassia Butyracea Seed Butter, Bassia Latifolia Seed Butter, Brassica Campestris (Rapeseed) Seed Oil, Buffalo Fat, Butter, Butyrospermum Parkii (Shea Butter), Butyrospermum Parkii (Shea Butter) Oil, C10-18 Triglycerides, C12-18 Acid Triglyceride, C8-12 Acid Triglyceride, Calendula Officinalis Seed Oil, Calophyllum Tacamahaca Seed Oil, Camellia Japonica Seed Oil, Camellia Kissi Seed Oil, Camellia Sinensis Seed Oil, Canola Oil, Caprylic/Capric Triglyceride, Caprylic/Capric/Lauric Triglyceride, Caprylic/Capric/Linoleic Triglyceride, Caprylic/Capric/Myristic/Stearic Triglyceride, Caprylic/Capric/Stearic Triglyceride, Carthamus Tinctorius (Safflower) Seed Oil, Carya Illinoensis (Pecan) Seed Oil, Caryocar Coriaceum Seed Oil, Castor Oil Benzoate, Cibotium Barometz Oil, Citrullus Vulgaris (Watermelon) Seed Oil, Cocos Nucifera (Coconut) Oil, Cod Liver Oil, Cod Liver/Mink/Tallow Triglyceride, Coix Lacryma-Jobi (Job's Tears) Seed Oil, Corylus Americana (Hazel) Seed Oil, Crambe Abyssinica Seed Oil, Cucurbita Pepo (Pumpkin) Seed Oil, Deer Fat, Elaeis Guineensis (Palm) Kernel Oil, Elaeis Guineensis (Palm) Oil, Emu Oil, Euterpe Oleracea Fruit Oil, Fish Liver Oil, Garcinia Indica Seed Butter, Gevuina Avellana Oil, Glycine Soja (Soybean) Lipids, Glycine Soja (Soybean) Oil, Goat Butter, Goat Fat, Gossypium Herbaceum (Cotton) Seed Oil, Helianthus Annuus (Sunflower) Seed Oil, Hippophae Rhamnoides Oil, Horse Fat, Hydrogenated Apricot Kernel Oil, Hydrogenated C12-18 Triglycerides, Hydrogenated Canola Oil, Hydrogenated Castor Oil, Hydrogenated Castor Oil Laurate, Hydrogenated Castor Oil Triisostearate, Hydrogenated Coconut Oil, Hydrogenated Cottonseed Oil, Hydrogenated Fish Oil, Hydrogenated Lard, Hydrogenated Menhaden Oil, Hydrogenated Milk Lipids, Hydrogenated Mink Oil, Hydrogenated Olive Oil, Hydrogenated Orange Roughy Oil, Hydrogenated Ostrich Oil, Hydrogenated Palm Kernel Oil, Hydrogenated Palm Oil, Hydrogenated Peanut Oil, Hydrogenated Pistachio Seed Oil, Hydrogenated Rapeseed Oil, Hydrogenated Rice Bran Oil, Hydrogenated Safflower Seed Oil, Hydrogenated Shark Liver Oil, Hydrogenated Shea Butter, Hydrogenated Soybean Oil, Hydrogenated Sunflower Seed Oil, Hydrogenated Sweet Almond Oil, Hydrogenated Tallow, Hydrogenated Vegetable Oil, Hydrogenated Wheat Germ Oil, Irvingia Gabonensis Kernel Butter, Isatis Tinctoria Seed Oil, Juglans Regia (Walnut) Seed Oil, Kyounin Yu, Lard, Lauric/Palmitic/Oleic Triglyceride, Limnanthes Alba (Meadowfoam) Seed Oil, Linum Usitatissimum (Linseed) Seed Oil, Luffa Cylindrica Seed Oil, Lupinus Albus Seed Oil, Macadamia Integrifolia Seed Oil, Macadamia Ternifolia Seed Oil, Mangifera Indica (Mango) Seed Butter, Mangifera Indica (Mango) Seed Oil, Marmot Oil, Melia Azadirachta Seed Oil, Melissa Officinalis Seed Oil, Menhaden Oil, Mink Oil, Moringa Pterygosperma Seed Oil, Mortierella Oil, Natane Yu, Neatsfoot Oil, Nigella Sativa Seed Oil, Oenocarpus Bacaba Fruit Oil, Oenocarpus Bataua Fruit Oil, Olea Europaea (Olive) Fruit Oil, Olea Europaea Olive) Husk Oil, Oleic/Linoleic Triglyceride, Oleic/Palmitic/Lauric/Myristic/Linoleic Triglyceride, Omental Lipids, Orange Roughy Oil, Orbignya Cohune Seed Oil, Orbignya Oleifera Seed Oil, Oryza Sativa (Rice) Bran Oil, Oryza Sativa (Rice) Germ Oil, Ostrich Oil, Papaver Orientale (Poppy) Seed Oil, Passiflora Edulis Seed Oil, Persea Gratissima (Avocado) Butter, Persea Gratissima (Avocado) Oil, Pinus Koraiensis Seed Oil, Pistacia Vera Seed Oil, Prunus Amygdalus Dulcis (Sweet Almond) Oil, Prunus Armeniaca (Apricot) Kernel Oil, Prunus Avium (Sweet Cherry) Seed Oil, Prunus Domestica Seed Oil, Prunus Insititia Seed Oil, Prunus Persica (Peach) Kernel Oil, Rabbit Fat, Ricinoleic/Caproic/Caprylic/Capric Triglyceride, Ricinus Communis (Castor) Seed Oil, Rosa Eglentaria Seed Oil, Salmon Oil, Salvia Hispanica Seed Oil, Santalum Album (Sandalwood) Seed Oil, Schleichera Trijuga Seed Oil, Sesamum Indicum (Sesame) Seed Oil, Shark Liver Oil, Shorea Robusta Seed Butter, Solanum Lycopersicum (Tomato) Fruit Oil, Solanum Lycopersicum (Tomato) Seed Oil, Tall Oil, Tallow, Telphairia Pedata Oil, Theobroma Cacao (Cocoa) Seed Butter, Theobroma Grandiflorum Seed Butter, Triarachidin, Tribehenin, Tricaprin, Tricaprylin, Trichodesma Zeylanicum Oil, Trierucin, Triethylhexanoin, Triheptanoin, Triheptylundecanoin, rihydroxymethoxystearin, Trihydroxystearin, Triisononanoin, Triisopalmitin, Triisostearin, Trilaurin, Trilinolein, Trilinolenin, Trimyristin, Triolein, Tripalmitin, Tripalmitolein, Triricinolein, Trisebacin, Tristearin, Triticum Vulgare (Wheat) Bran Lipids, Triticum Vulgare (Wheat) Germ Oil, Triundecanoin, Turtle Oil, Vaccinium Macrocarpon (Cranberry) Seed Oil, Vateria Indica Seed Butter, Virola Sebifera Nut Oil, Zea Mays (Corn) Germ Oil, Zea Mays (Corn) Oil.
Acetylated Hydrogenated Castor Oil, Actinidia Chinensis (Kiwi) Seed Oil, Adansonia Digitata Seed Oil, Argania Spinosa Kernel Oil, Argemone Mexicana Oil, Astrocaryum Murumuru Butter, Bertholletia Excelsa Seed Oil, Bixa Orellana Seed Oil, Borago Officinalis Seed Oil, Brain Lipids, Brassica Oleracea Italica (Broccoli) Seed Oil, C10-40 Isoalkyl Acid Triglyceride, Calodendrum Capense Nut Oil, Calophyllum Tacamahaca Seed Oil, Camellia Kissi Seed Oil, Carica Papaya Seed Oil, Caryocar Coriaceum Seed Oil, Castor Oil Benzoate, Chenopodium Quinoa Seed Oil, Citrullus Lanatus (Watermelon) Seed Oil, Cod Liver/Mink/Tallow Triglyceride, Coix Lacryma-Jobi (Job's Tears) Seed Oil, Cucumis Sativus (Cucumber) Oil, Daucus Carota Sativa (Carrot) Seed Oil, Glyceryl Triacetyl Hydroxystearate, Glyceryl Triacetyl Ricinoleate, Haematococcus Pluvialis Oil, Hydrogenated Egg Oil, Hydrogenated Evening Primrose Oil, Hydrogenated Grapeseed Oil, Hydrogenated Hazelnut Oil, Hydrogenated Hemp Oil, Hydrogenated Hemp Seed Oil, Hydrogenated Macadamia Seed Oil, Hydrogenated Sesame Seed Oil, Isatis Tinctoria Seed Oil, Lithospermum Officinale Seed Oil, Lupinus Albus Seed Oil, Lycium Barbarum Seed Oil, Mamushi Oil, Marrow Lipids, Milk Lipids, Morinda Citrifolia Seed Oil, Mucor Circinelloides Oil, Mustelic/Palmitic Triglyceride, Odontella Aurita Oil, Orbignya Speciosa Kernel Oil, Oxycoccus Palustris Seed Oil, Papaver Somniferum Seed Oil, Parinari Curatellifolia Seed Oil, Passiflora Edulis Seed Oil, Pinus Parviflora Seed Oil, Pinus Pentaphylla Seed Oil, Plukenetia Volubilis Seed Oil, Prunus Insititia Seed Oil, Punica Granatum Seed Oil, Pycnanthus Angolensis Nut Oil, Pyrus Malus (Apple) Seed Oil, Ribes Nigrum (Black Currant) Seed Oil, Rosa Moschata Seed Oil, Rosa Rubiginosa Seed Oil, Rubus Glaucus Seed Oil, Rubus Idaeus (Raspberry) Seed Oil, Schinziophyton Rautanenii Kernel Oil, Sclerocarya Birrea Seed Oil, Silkworm Chrysalis Oil, Soybean Glycerides, Spinal Cord Lipids, Sterculia Africana Seed Oil, Torreya Nucifera Seed Oil, Vaccinium Vitis-Idaea Seed Oil, Vanilla Planifolia Fruit Oil, Virola Sebifera Nut Oil.
Fatty Alcohols: Arachidyl Alcohol, Behenyl Alcohol, C9-11 Alcohols, C12-13 Alcohols, C12-15 Alcohols, C12-16 Alcohols, C14-15 Alcohols, C14-22 Alcohols, C20-22 Alcohols, Caprylic Alcohol, Cetearyl Alcohol, Cetyl Alcohol, Coconut Alcohol, C6-12 Perfluoroalkylethanol, Decyl Alcohol, Hydrogenated Jojoba Alcohol, Hydrogenated Rapeseed Alcohol, Hydrogenated Tallow Alcohol, Jojoba Alcohol, Lauryl Alcohol, Myristyl Alcohol, Oleyl Alcohol, Olive Alcohol, Palm Alcohol, Palm Kernel Alcohol, Stearyl Alcohol, Tallow Alcohol, Tridecyl Alcohol.
Ethers: Butoxyethyl Stearate, C12-14 Hydroxyalkyl Hydroxydimerdilinoleyl Ether, C12-14 Hydroxyalkyl Maltitol Ether, C16-18 Hydroxyalkyl Hydroxydimerdilinoleyl Ether, Cetyl Glyceryl Ether, Cetyloxypropyl Glyceryl Methoxypropyl Myristamide, Chimyl Alcohol, Chimyl Isostearate, Chimyl Stearate, Dicaprylyl Ether, Diretinyl Ether, Ethoxydiglycol Behenate, Ethoxydiglycol Isostearate, Ethoxydiglycol Oleate, Ethylhexyloxyglyceryl Palmitate, Eugenyl Glucoside, Hydrolyzed Proanthocyanidin, Hydroxyethyl Palmityl Oxyhydroxypropyl Palmitamide, Isopolyglycerin-3 Dimethicone, Isopropyl Hydroxycetyl Ether, Isostearyl Ferulate, Isostearyl Glyceryl Ether, Isostearyl Glyceryl Pentaerythrityl Ether, Lauryl Polyglyceryl-6 Cetearyl Glycol Ether, Oleyl Glyceryl Ether, Polyglyceryl-2 Lanolin Alcohol Ether, Polyglyceryl-2 Oleyl Ether, Polyglyceryl-3 Decyltetradecyl Ether, Polyglyceryl-3 Hydroxylauryl Ether, Polyglyceryl-4 Lauryl Ether, Polyglyceryl-4 Oleyl Ether, PPG-9 Diglyceryl Ether, Propylene Glycol Myristyl Ether.
Butoxyethyl Acetate, Diethoxydiglycol, Dimethoxydiglycol, Dimethyl Ether, Dimethyl Isosorbide, Dioxolane, Dipropylene Glycol, Dipropylene Glycol Dimethyl Ether, Ethoxydiglycol Acetate, Ethoxyethanol Acetate, Ethyl Ether, Ethyl Perfluorobutyl Ether, Ethyl Perfluoroisobutyl Ether, Methoxydiglycol, Methoxyethanol Acetate, Methoxyisopropyl Acetate, Methyl Hexyl Ether, Methyl Perfluorobutyl Ether, Methyl Perfluoroisobutyl Ether, Methylal, Propylene Glycol tert-Butyl Ether.

### Heterocyclic Compounds: Caprylyl Pyrrolidone, Lauryl Pyrrolidone, Myristyl Nicotinate, Pyridoxine Dicaprylate, Pyridoxine Dilaurate, Pyridoxine Dioctenoate, Pyridoxine Dipalmitate.

Useful systems are, for example, oils in the form of sprays or as liquid. Silicone oils may also be used in semisolid form, in the form of “silicone gels”. A further variant is oleogels which contain thickened triglycerides. Likewise suitable are water-free powders and aerosols.

Of particular suitability, therefore, for incorporating the present particles are essentially anhydrous formulations or systems in which the water-containing phase and the active ingredient phase are not in direct contact with one another and are only mixed together upon application (2-chamber or multichamber systems).

The customary cosmetic carriers or auxiliaries which are present in the cosmetic composition according to the invention include various oil components, such as fats, oils, silicone oil etc., further substances such as allantoin, thickeners, water-free preservatives, perfume oils, antioxidants, for example vitamin E, carotinoids or HALS (hindered amine light stabilizer) compounds.

Moreover, the method according to the invention may comprise a further UV protection substance, e.g. selected from the following classes:
1. p-aminobenzoic acid derivatives, for example 2-ethylhexyl 4-dimethylaminobenzoate;
2. salicylic acid derivatives, for example 2-ethylhexyl salicylate;
3. benzophenone derivatives, for example 2-hydroxy-4-methoxybenzophenone and its 5-sulfonic acid derivative;
4. dibenzoylmethane derivatives, for example 1-(4-tert-butylphenyl)-3-(4-methoxyphenyl)propane-1,3-dione;
5. diphenyl acrylates, for example 2-ethylhexyl 2-cyano-3,3-diphenyl acrylate and 3-benzofuranyl 2-cyanoacrylate;
6. 3-imidazol-4-ylacrylic acid and esters;
7. benzofuran derivatives, in particular 2-(p-aminophenyl)benzofuran derivatives, described in EP-A-582,189, U.S. Pat. No. 5,338,539, U.S. Pat. No. 5,518,713 and EP-A-613,893;
8. polymeric UV absorbers, for example the benzylidene malonate derivatives described in EP-A-709,080;
9. cinnamic acid derivatives, for example the 2-ethylhexyl 4-methoxycinnamate or isoamyl 4-methoxycinnamate disclosed in U.S. Pat. No. 5,601,811 and WO 97/00851;
10. camphor derivatives, for example 3-(4′-methyl)benzylidenebornan-2-one, 3-benzylidenebornan-2-one, N-[2(and 4)-2-oxyborn-3-ylidenemethyl)benzyl]acrylamide polymer, 3-(4′-trimethylammonium)benzylidenebornan-2-one methylsulfate, 3,3′-(1,4-phenylenedimethine)bis(7,7-dimethyl-2-oxobicyclo[2.2.1]heptan-1-methanesulfonic acid) and salts, 3-(4′-sulfo)benzylidenebornan-2-one and salts;
11. trianilino-s-triazine derivatives, for example 2,4,6-trianilino(p-carbo-2′-ethyl-1′-oxy)-1,3,5-triazine, and the UV absorbers disclosed in U.S. Pat. No. 5,332,568, EP-A-517,104, EP-A-507,691, WO 93/17002 and EP-A-570,838;
12. 2-hydroxyphenylbenzotriazole derivatives;
13. hydroxyphenyl-s-triazine compounds, for example the compounds described in U.S. Pat. No. 5,955,060;
14. 2-phenylbenzimidazole-5-sulfonic acid and salts thereof;
15. menthyl o-aminobenzoate;
16. TiO₂ (with various coatings), ZnO and mica.

Preferably, the inventive method comprises additional compounds selected from the group consisting of fats, oils, allantoin, thickeners, water-free preservatives, perfume oils, antioxidants, UV absorbers, amino acids, soluble tanning substances, precursors or encapsulated forms of a self tanning substance, dispersing agents and penetration aids.

Of interest is a method wherein the particles are crystalline or amorphous and have an average diameter between the low micrometer and the nanoscale range. Especially preferred is a method of the invention wherein 90% by weight or more of the particles are in the range between 10 micrometer and 1 nm.

The invention pertains also to a composition comprising
a) in an amount of 30-60% by weight of the composition a solid compound of the formula I, and
b) an organic liquid of low polarity or non-polar organic liquid.

Preferably, the composition comprises further components selected from the group consisting of fats, oils, allantoin, thickeners, water-free preservatives, perfume oils, antioxidants, UV absorbers, amino acids, soluble tanning substances, precursors or encapsulated forms of a self tanning substance, dispersing agents and penetration aids.

Of special interest is a composition wherein the UV absorber is a micronized UV absorber.

Preference is given to the use of mixing ratios of the present tanning substance and one or more optional further light-protective agents (e.g. as described in the below tables) from 1:99 to 99:1, preferably from 1:95 to 95:1 and most preferably from 10:90 to 90:10, based on weight. Of special interest are mixing ratios of from 20:80 to 80:20, preferably from about 40:60 to about 60:40 and most preferably approximately 50:50. Such mixtures can be used, inter alia, to improve the tanning effect, the sun protection or to increase UV absorption.

TABLE 1
Suitable UV filter substances which can be additionally
used with the compounds of the formula I or II or III

| | |
|---|---|
| DE 10013318 | T 1 pp 8-9, all Examples pp 10-13, T 2 pp 13-14, all Examples p 14, |
| | Ex A, B, C, D, E, F pp 19-20 |
| DE 10206562 A1 | Ex 1-3 p 10, Ex 4-7 p 11, Ex 8-15 pp 12-14 |
| DE 10238144 A1 | Ex on p 3-5; |
| DE 10331804 | T 1 p 4, T2 + 3 p 5 |
| DE 19704990 A1 | Ex 1-2 on pp 6-7; |
| EP 613 893 | Ex 1-5 + 15, T 1, pp 6-8 |
| EP 0 998 900 A1 | Ex on pp 4-11 |
| EP 1 000 950 | Comp. In Table 1, pp 18-21 |
| EP 1 005 855 | T3, p 13 |
| EP 1 008 586 | Ex 1-3, pp 13-15 |
| EP 1 008 593 | Ex 1-8, pp 4-5 |
| EP 1 027 883 | Compound VII, p 3 |
| EP 1 027 883 | Comp I-VI, p 3 |
| EP 1 028 120 | Ex 1-5, pp 5-13 |
| EP 1 059 082 | Ex 1;T 1, pp 9-11 |
| EP 1 060 734 | T 1-3, pp 11-14 |
| EP 1 064 922 | Compounds 1-34, pp 6-14 |
| EP 1 077 246 A2 | Ex 1-16 on pp 5-11; |
| EP 1 081 140 | Ex 1-9, pp 11-16 |
| EP 1 103 549 | Compounds 1-76, pp 39-51 |
| EP 1 108 712 | 4,5-Dimorph olino-3-hydroxypyridazine |
| EP 1 123 934 | T 3, p 10 |
| EP 1 129 695 | Ex 1-7, pp 13-14 |
| EP 1 167 359 | Ex 1, p 11 and Ex 2, p 12 |
| EP 1 232 148 B1 | Ex 4-17 on pp 3-5; |
| EP 1 258 481 | Ex 1, pp 7,8 |
| EP 1 310 492 A1 | Ex 1-16 on pp 22-30 |
| EP 1 371 654 A1 | Ex on pp 5-7 |
| EP 1 380 583 A2 | Ex 1, p 6; |
| EP 1 423 351 A2 | Ex 1-16 on pp 31-37; |
| EP 1 423 371 A1 | T 1 on pp 4-8, Ex on p 9, Ex 1-9 on pp 36-42; |
| EP 1 454 896 A1 | Ex 1-5 on pp 10-13, Examples on pp 4-5; |
| EP 1 471 059 A1 | Ex 1-5 on pp 4-5; |
| EP 1484051 A2 | Formula III-VII on pp 18-19, Ex 7-14 on pp 7-9, Ex 18-23 on pp 11- |
| | 12, Ex 24-40 on pp 14-17; |
| EP 420 707 B1 | Ex 3, p 13 (CAS Reg. No 80142-49-0) |
| EP 503 338 | T 1, pp 9-10 |
| EP 517 103 | Ex 3, 4, 9, 10 pp 6-7 |
| EP 517 104 | Ex 1, T 1, pp 4-5; Ex 8, T 2, pp 6-8 |
| EP 626 950 | All compounds |
| EP 669 323 | Ex 1-3, p 5 |
| EP 743 309 A1 | Ex 1-12 on pp 18-24; |
| EP 780 382 | Ex 1-11, pp 5-7 |
| EP 823 418 | Ex 1-4, pp 7-8 |
| EP 826 361 | T 1, pp 5-6 |
| EP 832 641 | Ex 5 + 6 p 7; T 2, p 8 |
| EP 832 642 | Ex 22, T3, pp 10-15; T 4, p 16 |
| EP 852 137 | T 2, pp 41-46 |
| EP 858 318 | T 1, p 6 |
| EP 863 145 | Ex 1-11, pp 12-18 |
| EP 878 469 A1 | T 1, pp 5-7; |
| EP 895 776 | Comp. In rows 48-58, p 3; R 25 + 33, p 5 |
| EP 911 020 | T 2, pp 11-12 |
| EP 916 335 | T 2-4, pp 19-41 |
| EP 924 246 | T 2, p 9 |
| EP 933 376 | Ex 1-15, pp 10-21 |
| EP 944 624 | Ex 1 + 2, pp 13-15 |
| EP 945 125 | T 3 a + b, pp 14-15 |
| EP 95 097 | Ex 1, p 4 |
| EP 967 200 | Ex 2; T 3-5, pp 17-20 |
| EP 969 004 | Ex 5, T 1, pp 6-8 |
| FR 2842806 A1 | Ex I p 10, Ex II p 12 |
| FR 2861075 A1 | Ex 1-3 on pp 12-14; |
| FR 2862641 | Formula 3 on p 4; Ex A-J on pp 7-9; |
| KR 2004025954 | All kojyl benzoate derivatives |
| JP 06135985 A2 | Formula 1 on p 2; Ex 1-8 on pp 7-8; |
| JP 2000319629 | CAS Reg Nos. 80142-49-0, 137215-83-9, 307947-82-6 |
| JP 2003081910 A | Ex on p 1; |
| JP 3686911 B2 | All benzylidene-gamma-butyrolactone derivatives |
| US 2003/0053966A1 | Ex on pp 3-6 |
| US 2004057912 A1 | Ex on p 7-9, Ex 1 on p 10; |
| US 2004057914 A1 | Ex on p 8-12, Ex 1 on p 12; |
| US 2004/0057911A1 | Formula I and II on p 1; formula III and IV on p3; Ex 1-3 on pp 5-6; |
| US 2004/0071640A1 | Ex 1-12 on pp 4-7; |
| US 2004/0091433A1 | Ex 1-6 on pp 14-16; |
| US 2004/0136931A1 | Ex 1-3 on p 7; |
| US 2004/0258636A1 | Ex 1-11 on pp 9-15; |
| US 2005/0019278A1 | Ex 1-9 on pp 6-8; |
| US 2005/0136012A1 | Formula 1 on p 2; |
| US 2005/0136014A1 | Formula a-c on p 2; Examples on p 3; |
| US 2005/0201957A1 | Formula 1 on p 1; Ex A, B, C, D, E, F, G on pp 2-3; |
| US 2005/0249681A1 | All compounds on pp 2-3, Ex 1 on p 6; |
| U.S. Pat. No. 5,635,343 | All compounds on pp 5-10 |
| U.S. Pat. No. 5,332,568 | Ex 1, p 5, T 1 + 2, pp 6-8 |
| U.S. Pat. No. 5,338,539 | Ex 1-9, pp 3 + 4 |
| U.S. Pat. No. 5,346,691 | Ex 40, p 7; T 5, p 8 |
| U.S. Pat. No. 5,801,244 | Ex 1-5, pp 6-7 |
| U.S. Pat. No. 6,613,340 | Ex I, II pp 9-11, Examples on rows 28-53 p 6 |
| US 6 800 274 B2 | Formulas I-VI and IX-XII on pp 14-18; |
| US 6 890 520 B2 | Ex 1-10 on pp 6-9; |
| US 6926887 B2 | Ex A on pp 5/6; Formulas I-VIII on pp 27-29; |
| US 6936735 B2 | Formula 1-2 on p 2; formula 3-4 on p 6; |
| WO 0149686 | Ex 1-5, pp 16-21 |
| WO 0168047 | Tables on pp 85-96 |
| WO 0181297 | Ex 1-3, pp 9-11 |
| WO 0191695 | Formula I on p 4, T on p 8 |
| WO 0202501 A1 | Ex Ia-c, p 5 |
| WO 02069926 A1 | Ex on p 9, Ex on pp 17-23 |
| WO 02072583 | T on pp 68-70 |
| WO 02080876 | Ex 1 on pp 7-9 |
| WO 0238537 | All compounds p 3, compounds on rows 1-10 p 4 |
| WO 03004557 A1 | Ex A1-A29 on pp 36-57; |
| WO 03007906 | Ex I-XXIII, pp 42-48 |
| WO 03086341 A2 | Formula 2-21, pp 4-6; |
| WO 03092643 A1 | T on pp 34-35, compounds listed on p 16 |
| WO 03097577 A1 | Ex on pp 6-8; Ex 1-3 on pp 15-18; |
| WO 03104183 A1 | Formula I-IV on p 1; Ex 1-5 on pp 27-28; |
| WO 04000256 A1 | Ex 1-10 on pp 18-24 |
| WO 04020398 A1 | Ex 1-3 on pp 14-17 |
| WO 04020398 A1 | Formulas I-VI on pp 21-24, Formula IX on p 25; |
| WO 05009938 A2 | Formula I on p 1; Ex 1-2 on pp 14-15; |
| WO 05065154 A2 | Formula a-c on pp 5-6; |
| WO 05080341 A1 | Formula 1 on p 3; Examples on pp 9-13; |
| WO 9217461 | Ex 1-22, pp 10-20 |
| WO 9220690 | Polymeric Comp in Examples 3-6 |
| WO 9301164 | T 1 + 2, pp 13-22 |
| WO 9714680 | Ex 1-3, p 10 |

(Abbreviations T: Table, R: row, Comp: compound, Ex: compound(s) of Patent Example, p: page; the generic scope of the UV absorbers is described in the left-hand column; specific compounds are indicated in the right-hand column)

TABLE 2
Suitable UV filter substances and adjuvants which can be additionally
used with the compounds of the formula I or II or III

| No. | Chemical Name | CAS No. |
|---|---|---|
| 1 | (+/−)-1,7,7-trimethyl-3-[(4-methylphenyl)methylene]bicyclo- | 36861-47-9 |
| | [2.2.1]heptan-2-one; p-methyl benzylidene camphor | |
| 2 | 1,7,7-trimethyl-3-(phenylmethylene)bicyclo[2.2.1]heptan-2-one; | 15087-24-8 |
| | benzylidene camphor | |
| 3 | (2-Hydroxy-4-methoxyphenyl)(4-methylphenyl)methanone | 1641-17-4 |
| 4 | 2,4-dihydroxybenzophenone | 131-56-6 |
| 5 | 2,2′,4,4′-tetrahydroxybenzophenone | 131-55-5 |
| 6 | 2-Hydroxy-4-methoxy benzophenone; | 131-57-7 |
| 7 | 2-Hydroxy-4-methoxy benzophenone-5-sulfonic acid | 4065-45-6 |
| 8 | 2,2′-dihydroxy-4,4′-dimethoxybenzophenone | 131-54-4 |
| 9 | 2,2′-Dihydroxy-4-methoxybenzophenone | 131-53-3 |
| 10 | Alpha-(2-oxoborn-3-ylidene)toluene-4-sulphonic acid and its salts (Mexoryl SL) | 56039-58-8 |
| 11 | 1-[4-(1,1-dimethylethyl)phenyl]-3-(4-methoxyphenyl)propane-1,3-dione (Avobenzone) | 70356-09-1 |
| 12 | Methyl N,N,N-trimethyl-4-[(4,7,7-trimethyl-3-oxobicyclo[2,2,1]hept-2- | 52793-97-2 |
| | ylidene)methyl]anilinium sulphate (Mexoryl SO) | |
| 22 | 3,3,5-Trimethyl cyclohexyl-2-hydroxy benzoate; homosalate | 118-56-9 |
| 23 | Isopentyl p-methoxycinnamate; isoamyl methoxy cinnamate | 71617-10-2 |
| 27 | Menthyl-o-aminobenzoate | 134-09-8 |
| 28 | Menthyl salicylate | 89-46-3 |
| 29 | 2-Ethylhexyl 2-cyano,3,3-diphenylacrylate; octocrylene | 6197-30-4 |
| 30 | 2-ethylhexyl 4-(dimethylamino)benzoate | 21245-02-3 |
| 31 | 2-ethylhexyl 4-methoxycinnamate; octyl methoxy cinnamate | 5466-77-3 |
| 32 | 2-ethylhexyl salicylate | 118-60-5 |
| 33 | Benzoic acid,4,4′,4″-(1,3,5-triazine-2,4,6-triyltriimino)tris-,tris(2- | 88122-99-0 |
| | ethylhexyl)ester; 2,4,6-Trianilino-(p-carbo-2′-ethylhexyl-1′-oxi)-1,3,5- | |
| | triazine (Octyl Triazone) | |
| 34 | 4-aminobenzoic acid | 150-13-0 |
| 35 | Benzoic acid, 4-amino-, ethyl ester, polymer with oxirane | 113010-52-9 |
| 38 | 2-phenyl-1H-benzimidazole-5-sulphonic acid; | 27503-81-7 |
| | phenylbenzimidazolsulfonic acid | |
| 39 | 2-Propenamide, N-[[4-[(4,7,7-trimethyl-3-oxobicyclo[2.2.1]hept-2- | 147897-12-9 |
| | ylidene)methyl]phenyl]methyl]-, homopolymer | |
| 40 | Triethanolamine salicylate | 2174-16-5 |
| 41 | 3,3′-(1,4-phenylenedimethylene)bis[7,7-dimethyl-2-oxo- | 90457-82-2 |
| | bicyclo[2.2.1]heptane-1 methanesulfonic acid] (Cibafast H) | |
| 42 | Titanium dioxide (primary particle size 10-50 nm) | 13463-67-7 |
| | For example T805 or Eusolex T-AVO, Eusolex T-2000, | |
| | Titaniumdioxid VT 817 | |
| 44 | Zinc oxide (primary particle size 20-100 nm) | 1314-13-2 |
| | For example Zinc oxide NDM, Zinc oxide Z-Cote HP1, Nanox Zinc | |
| | oxide | |
| 45 | 2,2′-Methylene-bis-[6-(2H-benzotriazol-2-yl)-4-(1,1,3,3-tetramethyl- | 103597-45-1 |
| | butyl)-phenol] (Tinosorb M) | |
| 46 | 2,4-bis{[4-(2-ethylhexyloxy)-2-hydroxy]-phenyl}-6-(4-methoxyphe- | 187393-00-6 |
| | nyl)-(1,3,5)-triazine (Tinosorb S) | |
| 47 | 1H-Benzimidazole-4,6-disulfonic acid, 2,2′-(1,4-phenylene)bis-, di- | 180898-37-7 |
| | sodium salt | |
| 48 | Benzoic acid, 4,4′-[[6-[[4-[[(1,1-dimethylethyl)amino]carbonyl]- | 154702-15-5 |
| | phenyl]amino]1,3,5-triazine-2,4-diyl]diimino]bis-, bis(2-ethylhexyl)- | |
| | ester; diethylhexyl butamido triazone (Uvasorb HEB) | |
| 49 | Phenol, 2-(2H-benzotriazol-2-yl)-4-methyl-6-[2-methyl-3-[1,3,3,3- | 155633-54-8 |
| | tetramethyl-1-[(trimethylsilyl)oxy]disiloxanyl]propyl]-; drometrizole | |
| | trisiloxane (Mexoryl XL) | |
| 50 | Dimethicodiethylbenzalmalonate; Polysilicone 15 (Parsol SLX) | 207574-74-1 |
| 51 | Benzenesulfonic acid, 3-(2H-benzotriazol-2-yl)-4-hydroxy-5-(1- | 92484-48-5 |
| | methylpropyl)-, monosodium salt (Tinogard HS) | |
| 52 | Benzoic acid, 2-[4-(diethylamino)-2-hydroxybenzoyl]-, hexyl ester | 302776-68-7 |
| | (Uvinul A Plus) | |
| 53 | 1-Dodecanaminium, N-[3-[[4-(dimethylamino)benzoyl]amino]propyl]- | 156679-41-3 |
| | N,N-dimethyl-, salt with 4-methylbenzenesulfonic acid (1:1) (Escalol | |
| | HP610) | |
| 54 | 1-Propanaminium, N,N,N-trimethyl-3-[(1-oxo-3-phenyl-2-propenyl)- | 177190-98-6 |
| | amino]-, chloride | |
| 55 | 1H-Benzimidazole-4,6-disulfonic acid, 2,2′-(1,4-phenylene)bis- | 170864-82-1 |
| 56 | 1,3,5-Triazine, 2,4,6-tris(4-methoxyphenyl)- | 7753-12-0 |
| 57 | 1,3,5-Triazine, 2,4,6-tris[4-[(2-ethylhexyl)oxy]phenyl]- | 208114-14-1 |
| 58 | 1-Propanaminium, 3-[[3-[3-(2H-benzotriazol-2-yl)-5-(1,1-dimethyl- | 340964-15-0 |
| | ethyl)-4-hydroxyphenyl]-1-oxopropyl]amino]-N,N-diethyl-N-methyl-, | |
| | methyl sulfate (salt) | |
| 59 | 2-Propenoic acid, 3-(1H-imidazol-4-yl)- | 104-98-3 |
| 60 | Benzoic acid, 2-hydroxy-, [4-(1-methylethyl)phenyl]methyl ester | 94134-93-7 |
| 61 | 1,2,3-Propanetriol, 1-(4-aminobenzoate) (Glyceryl PABA) | 136-44-7 |
| 62 | Benzeneacetic acid, 3,4-dimethoxy-a-oxo- | 4732-70-1 |
| 63 | 2-Propenoic acid, 2-cyano-3,3-diphenyl-, ethyl ester | 5232-99-5 |
| 64 | Anthralinic acid, p-menth-3-yl ester | 134-09-8 |
| 65 | 2,2′-bis(1,4-phenylene)-1H-benzimidazole-4,6-disulphonic acid mo- | 349580-12-7, |
| | no sodium salt or Disodium phenyl dibenzimidazole tetrasulfonate | |
| | (Neo Heliopan AP) | |
| 66 | 1,3,5-Triazine-2,4,6-triamine, N,N′-bis[4-[5-(1,1-dimethylpropyl)-2- | 288254-16-0 |
| | benzoxazolyl]phenyl]-N″-(2-ethylhexyl)- (Uvasorb K2A) | |
| 67 | Merocyanine derivatives as described in WO 2004006878, | |
| | WO2006032741, IPCOM000022279D and in IP.COM JOURNAL | |
| | (2005), 5(7B), 18 | |
| 69 | sterols (cholesterol, lanosterol, phytosterols), as described in | |
| | WO0341675 | |
| 70 | mycosporines and/or mycosporine-like amino acids as described in | |
| | WO2002039974, e.g. Helioguard 365 from Milbelle AG, isolated | |
| | mycosporine like amino acids from the red alga porphyra umbilicalis | |
| | (INCI: Porphyra Umbilicalis) that are encapsulated into liposomes,) | |
| 71 | alpha-lipoic-acid as described in DE 10229995 | |
| 72 | synthetic organic polymers as described in EP 1371358, | |
| | [0033]-[0041] | |
| 73 | phyllosilicates as described in EP 1371357 [0034]-[0037] | |
| 74 | silica compounds as described in EP1371356, [0033]-[0041] | |
| 75 | inorganic particles as described in DE10138496 [0043]-[0055] | |
| 76 | latex particles as described in DE10138496 [0027]-[0040] | |
| 77 | 1H-Benzimidazole-4,6-disulfonic acid, 2,2′-(1,4-phenylene)bis-, | 180898-37-7 |
| | disodium salt; Bisimidazylate (Neo Heliopan APC) | |
| 82 | Di-2-ethylhexyl-3,5-dimethoxy-4-hydroxy-benzalmalonate (Oxynex | |
| | ST, EMD Chemicals, as described in US 20040247536) | |
| 83 | 2,4,6-Tris-1,1′,4′,1″-terphenyl-4-yl-1,3,5-triazine | |
| 84 | 2,4,6-Tris(p-biphenylyl)-s-triazine | 31274-51-8 |

The UV absorbers described in “Sunscreens”, Eds. N. J. Lowe, N. A. Shaath, Marcel Dekker, Inc., New York and Basle or in Cosmetics & Toiletries (107), 50 ff (1992) can also be used as additional UV protection substances in the cosmetic composition according to the invention.

UV filters can also be used in encapsulated form. Specifically, the following advantages arise:

The hydrophilicity of the capsule wall may be adjusted independently of the solubility of the UV filter. Thus, for example, even hydrophobic UV filters can be incorporated into purely aqueous preparations. In addition, the oily impression, often perceived as being unpleasant, upon application of the preparation comprising hydrophobic UV filters is suppressed. Certain UV filters, in particular dibenzoylmethane derivatives, exhibit only reduced photostability in cosmetic preparations. By encapsulating these filters or compounds which impair the photostability of these filters, such as, for example, cinnamic acid derivatives, it is possible to increase the photostability of the entire preparation. The literature discusses time and again the penetration of the skin by organic UV filters and the associated irritancy potential upon direct application to the human skin. The encapsulation of the corresponding substances that is proposed here suppresses this effect. It is therefore preferred according to the invention when one or more of the abovementioned UV filters are present in encapsulated form. In this connection, it is advantageous if the capsules are so small that they cannot be observed with the naked eye. To achieve the abovementioned effects, it is further necessary that the capsules are sufficiently stable and do not release the encapsulated active ingredient (UV filter), or release it only to a low degree, into the surrounding area.

To improve the shade of brown, the substances/formulations according to the invention can also be used together with an amino acid (e.g. tryptophan, cysteine, histidine, arginine, lysine, tyrosine, aspartic acid, methionine, proline, phenylalanine, hydroxyproline, leucine, glycine, isoleucine, threonine, serine, valine, alanine, cystine), a soluble tanning substance e.g. of the type initially mentioned, a precursor or encapsulated form of a self tanning substance such as disclosed in EP-A-796838, EP-A-710478, WO-A-01/085124, U.S. Pat. No. 6,007,796, WO-A-05/025531, and/or a penetration aid (e.g. dimethicone, a linear methylpoly-siloxane).

Advantageous tanning agents which may be used for the purposes of the present invention as additional self-tanning agents are the following substances:

5-hydroxy-1,4-naphthoquinone (juglone), 2-hydroxy-1,4-naphthoquinone (lawsone), 1,3-di-hydroxyacetone (DHA), erythrulose, bronzers, pigmentation agents (e.g., methoxsalen, trioxsalen, and melanin), dyes, botanical extracts (e.g., silver birch (Betulla alba), and Mahakanni STLC (Eclipta alba), tyrosine, alpha-hydroxy aldehydes and ketones, glyceraldehyde and related alcohol aldehydes, various indoles, imidazoles, methyl glyoxal, glycerol aldehyde, alloxan, 2,3-dihydroxysuccindialdehyde, 2,3-dimethoxysuccindialdehyde, 2-amino-3-hydroxy-succindialdehyde and 2-benzylamino-3-hydroxysuccindialdehyde.

Other sunless tanning agents, including those known and unknown to a person of skill in the art, are also contemplated as being useful with the present invention. Non-limiting examples of the sunless tanning agents are also described in U.S. Pat. Nos. 6,482,397; 6,262,541; 5,559,146; 6,447,760; and 6,443,164.

The concentration of the at least one additional self-tanning substance preferred according to the invention is in the range from 0.01 to 15 percent by weight, preferably in the range from 0.05 to 5 percent by weight and particularly preferably at most 1% by weight.

Further and more specific components, especially UV absorbers, silicones etc., useful in the present formulations are as disclosed in “Emollients in Personal Care for Solubilizing Organic UV Absorbers” (IPCOM000134094D) and “Tinogards as Skin Active” (IPCOM000130489D), accessible e.g. under: https://priorart.ip.com/.

For the preparation of the particle dispersions it is possible to use any known processes that are suitable for the preparation of microparticles, e.g.:
wet-grinding with a hard grinding medium, for example zirconium silicate and a protective surfactant or a protective polymer in water or in a suitable organic solvent;
spray-drying from a suitable solvent, for example aqueous suspensions or suspensions containing organic solvents, or true solutions in water, ethanol, dichloroethane, toluene or N-methylpyrrolidone etc.
by the expansion according to the RESS process (Rapid Expansion of Supercritical Solutions) of supercritical fluids (e.g. CO₂) in which the UV filter(s) is/are dissolved, or the expansion of fluid carbon dioxide together with a solution of one or more of the substances in a suitable organic solvent;
by reprecipitation from suitable solvents, including supercritical fluids (GASR process=Gas Anti-Solvent Recrystallisation/PCA process=Precipitation with Compressed Anti-solvents).

As grinding apparatus for the preparation of the present micronised substances according to the invention there may be used, for example, a jet mill, ball mill, vibratory mill or hammer mill, preferably a high-speed mixing mill. The grinding is preferably carried out with a grinding aid, for example an alkylated vinylpyrrolidone polymer, a vinylpyrrolidone/vinyl acetate copolymer, an acyl glutamate, an alkyl polyglucoside, ceteareth-25 or especially a phospholipid.

The invention therefore further pertains to a process for the preparation of a cosmetic formulation for the artificial tanning of the skin or the colouration of a keratinic fibre, characterized in that a solid compound of the formula I is subjected to wet-grinding, spray-drying, an expansion process using a supercritical fluid, or a reprecipitation, in an organic liquid of low polarity or non-polar organic liquid.

The particle size (diameter) is preferably from the low micrometer down to the nanoscale range; for example, 90% by weight or more of the particles are in the range between 10 micrometer and 1 nm, preferably in the range between 5 micrometer and 5 nanometer. The weight average particle size generally is in the nanoscale range, e.g. in the range from 5 to 1000 nm, preferably from 10 to 500 nm. The particles used in the present formulation are characterized by their x-ray diagram; they may be crystalline or amorphous.

For the final application, e.g. to the human skin or hair, the dispersion of the present invention usually is incorporated into a cosmetic formulation containing one or more further components.

The invention further pertains to a cosmetic formulation comprising
a) a solid particle of a tanning substance of a compound of the formula I according to claim 1 in dispersed form, and
b) a customary cosmetic carrier or auxiliary.

Of interest is a cosmetic formulation wherein component (a) is present in an amount of 1 to 15%, for example 2 to 10%, by weight of the total cosmetic formulation.

Of special interest is a cosmetic formulation in the form of a spray.

The instant application pertains also to the use of a dispersion of solid particles of a tanning substance of a compound of the formula I in an organic liquid of low polarity or non-polar organic liquid for the colouration of skin or a keratinic fibre, for the artificial tanning of the skin, for the levelling of differences in the brightness of the skin, for the protection of the skin against solar radiation, or for the colouration of human hair.

### EXAMPLES

### Preparation of a Cosmetic Formulation

A mixture of the following components is prepared:

25 parts by weight of crystalline compound of formula II; (dihydroxyacetone-dimer cosmetic use grade, MERCK, Germany)
2 parts by weight of Abil® EM-90 (INCI: Cetyl PEG/PPG-10/1 Dimethicone; Degussa Care Specialties)
2 parts by weight of Hostacerin® DGL (INCI: PEG-10 Polyglyceryl-2 Laurate; Clariant)
71 parts by weight of cyclomethicone (INCI: Cyclohexasiloxane; CAS-No. 540-97-6)

The slurry is ground in a ball mill to a particle size of the solid of 5-5000 nm.

The formulation thus obtained may be applied as such. Alternatively, it may be mixed with one or more further components, e.g. as mentioned further above.

### Application

Areas of 2 cm² are marked on the forearm of a number of test persons. The cosmetic formulation prepared in the above example is applied to the marked areas on the skin. The colour achieved is compared with the colour of the surrounding skin immediately after application and after the time periods indicated in the below table. During the time period of survey, the test person's forearm is kept covered and unexposed to any UV radiation. Results assessed visually are compiled in the following table.

TABLE
Tanning effect after application

| Test | Time period after application | | | | |
|---|---|---|---|---|---|
| person No. | Immediately | 3 hours | 24 hours | 3 days | 6 days |
| 1 | not detectable | slight | max. | deep | decreasing, well detectable |
| 2 | not detectable | slight | max. | deep | decreasing, well detectable |

The test shows that the present self-tanning preparation based on a dispersed solid as the tanning substance provides good results in the formation and duration of colour.

### Application Examples of Cosmetic Compositions

### Example 1

### Stick

| | Examples | | | | | | |
|---|---|---|---|---|---|---|---|
| Component [% b.w.] | A | b | c | d | e | f | g |
| Cyclopentasiloxane (and) Cyclohexasiloxane | 50% | — | — | 40% | 20% | — | 5% |
| Decamethylcyclopentasiloxane | — | 35% | 25% | — | 20% | — | — |
| Dodecamethylcyclohexasiloxane | — | 30% | 30% | — | 20% | — | — |
| Dimethicone | — | — | — | 25% | — | 5% | 5% |
| Stearyl alcohol | 25% | 25% | 25% | 22% | 23% | — | 5% |
| Glyceryl stearate (and) PEG-100 Stearate | — | — | — | 1% | 3% | — | — |
| Sodium Stearate | — | — | — | — | — | 10% | 10% |
| Isohexadecane | 5% | — | 5% | — | — | 5% | 5% |
| Isododecane | 5% | — | 5% | — | — | 5% | 5% |
| Hydrogenated castor oil | 1% | 1% | 1% | — | 5% | 5% | 5% |
| PEG-8 Caprylic/Capric Glycerides | — | — | — | — | — | 5% | 5% |
| PPG-14 butyl ether | 7% | 2% | 2% | — | 2% | — | — |
| PEG-75 Hydrogenated castor oil | — | — | — | 5% | 2% | 5% | 5% |
| Propylene Glycol | — | — | — | — | — | 30% | 30% |
| Alcohol Den. | — | — | — | — | — | 25% | 15% |
| Talc | 2% | 2% | 2% | 2% | — | — | — |
| Solid particles of tanning substance | 5% | 5% | 5% | 5% | 5% | 5% | 5% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |

### Example 2

### Clear Sun Stick

| | Examples | | | | | | |
|---|---|---|---|---|---|---|---|
| Component [% b.w.] | A | b | c | d | e | f | g |
| PPG-15 butyl ether | 40% | 20% | 10% | 30% | 25% | 35% | 15% |
| Stearyl Alcohol | 8% | 10% | 8% | 10% | 6% | 7% | 5% |
| Cyclopentasiloxane (and) Cyclohexasiloxane | 30% | 30% | 20% | 15% | 20% | 18% | 25% |
| PEG-4000 Hydrogenated castor oil | — | 5% | 8% | — | 5% | — | 10% |
| Glyceryl stearate (and) PEG-100 Stearate | 2% | 2% | 3% | 5% | 4% | 2.50% | 5% |
| Isohexadecane | — | — | 10% | 8% | — | 5% | — |
| Isododecane | — | 5% | — | 4% | 8% | — | 5% |
| Perfluorohexane | — | — | 5% | — | 4% | — | 6.50% |
| Perfluoromethylcyclopentane | — | 2.5% | 5% | — | — | 6.50% | — |
| Synthetic ester oils | — | 4.5% | 8.50% | — | — | 5% | 10% |
| Mineral, synthetic or silicone waxes | — | 1% | 4% | 8% | 10% | 5% | 2% |
| Bis-ethylhexyoxyphenol Methoxyphenyl | 3% | 3% | 2.50% | 4% | 2% | 4% | 2.50% |
| triazine | | | | | | | |
| Ethylhexyl Methoxycinnamate | 7% | 7% | 5% | 3% | 8% | 3% | 7% |
| Micronized inorganic sunscreen such as Tio2 | 5% | 5% | 6% | 8% | 5% | 7% | 7% |
| Solid particles of tanning substance | 5% | 5% | 5% | 5% | 3% | 2% | 1% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |

### Example 3

### Alcoholic Water Proof Sunscreen Gel

| | Examples | | | | | | |
|---|---|---|---|---|---|---|---|
| Component [% b.w.] | A | b | c | d | e | f | g |
| Ethylhexyl Salicylate | 6% | 6% | 6% | 6% | 5% | 3% | 4.50% |
| Bis-ethylhexyoxyphenol Methoxyphenyl | 2% | 2% | 2% | 2% | 4% | 3% | 1.50% |
| triazine | | | | | | | |
| Ethylhexyl Methoxycinnamate | 8% | 8% | 8% | 8% | 7% | 10% | 10% |
| PEG/PPG - 4/12 Dimethicone | 2% | — | — | — | — | 2% | 3% |
| PEG/PPG -18/1 Dimethicone | — | — | 2% | 2% | 5% | 3% | 4% |
| C12-15 alkyl benzoate | 12% | 10% | 10% | 10% | — | 5% | 10% |
| Cyclopentasiloxane (and) Cyclohexasiloxane | 16% | 10% | 10% | 10% | 10% | 25% | 15% |
| Acrylates/Octyl propenamide copolymer | 1% | — | — | — | — | 2% | 3% |
| SD alcohol | 45% | 50% | 50% | 50% | 60% | 40% | 35% |
| Hydroxypropyl Cellulose | 1% | 2% | 2% | 2% | 0.50% | — | 1% |
| Crosslinked elastomeric organopolysiloxanes | — | 5% | 5% | 5% | 3% | 5% | 8% |
| Olefin/Styrene copolymer | 2% | — | — | — | — | 2.50% | 2% |
| Hydrogenated Styrene/alkylene copolymer | — | 2% | 2% | — | — | — | 2% |
| Hydrogenated alkylene/Styren copolymer | — | — | — | 2% | 2.50% | 2.50% | — |
| Solid particles of tanning substance | 5% | 5% | 5% | 5% | 3% | 2% | 1% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |

### Example 4

### Sunscreen Oil Gel

| | Examples | | | | | | |
|---|---|---|---|---|---|---|---|
| Component [% b.w.] | A | b | c | d | e | f | g |
| Hydrogenated polyisobutene | 17% | 10% | 12% | 15% | 20% | 10% | 20% |
| Isohexadecane | — | 7% | 5% | 8% | — | 10% | 5% |
| Mineral oil (an) hydrogenated | 35% | 32% | 20% | 18% | 28% | 25% | 15% |
| butylene/ethylene/styrene copolymer (and) | | | | | | | |
| hydrogenated ethylene/propylene/styrene | | | | | | | |
| copolymer | | | | | | | |
| Olefin/Styrene copolymer | — | — | 4% | 5% | 3% | 4% | 2% |
| Phenyl Trimethicone | — | 10% | 15% | 20% | 15% | 20% | 15% |
| Octyldodecyl Neopentanoate | 15% | 10% | 15% | 13% | 15% | 10% | 20% |
| Tocopheryl acetate | 0.5% | 0.5% | 1% | 0.50% | 0.50% | 1% | — |
| C12-15 alkyl benzoate | 14.5 | 10% | 11.5% | 10% | 5% | 4% | 4% |
| Bis-ethylhexyoxyphenol Methoxyphenyl | 2% | 2% | 3% | 4% | 2.50% | 1.50% | 4% |
| triazine | | | | | | | |
| Ethylhexyl Methoxycinnamate | 8% | 8% | 7% | 6% | 8.5% | 9% | 8% |
| Silica Silylate | — | — | 3% | 4% | 2% | 1% | — |
| Silica | 2% | 2.5% | — | — | — | — | 3% |
| Bentonite | — | — | 2.50% | 1.50% | — | — | — |
| Quaternized Bentonite | — | — | — | — | — | 1.50% | 3% |
| Silica Dimethyl Silylate | — | 3% | — | — | 3% | 2.50% | — |
| Solid particles of tanning substance | 5% | 5% | 5% | 5% | 3% | 2% | 1% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |

### Example 5

### Oil Lotion

| | Examples | | | | | | |
|---|---|---|---|---|---|---|---|
| Component [% b.w.] | A | b | c | d | e | f | g |
| Hydrogenated Polyisobutene (and) | 40% | 40% | 25% | 30% | 20% | 35% | 40% |
| Hdrogenated butylene/ethylene/styrene | | | | | | | |
| copolymer (and) Hydrogenated | | | | | | | |
| ethylene/propylene/styrene copolymer | | | | | | | |
| Isopropyl palmitate | 30% | 25% | 20% | 25% | 30% | 20% | 15% |
| PEG-40 Sorbitan peroleate | — | — | 2% | 5% | — | 5% | — |
| PEG-8 laurate | 5% | — | 5% | — | 5% | 5% | 5% |
| Squalane | 5% | — | 5% | — | 7% | 10% | — |
| Hydrogenated polydecenes | — | 10% | 5% | — | 10% | 10% | 5% |
| Synthetic esters | 10% | — | 10% | 15% | 5% | 8% | 10% |
| Crosslinked elastomeric organopolysiloxanes | — | 5% | 3% | — | — | 2% | — |
| Isohexadecane | 5% | — | 15% | 10% | 5% | 5% | 14% |
| PPG-14 butyl ether | — | 5% | — | 10% | 7.50% | — | 7.50% |
| PEG-75 Hydrogenated castor oil | — | 10% | 5% | — | 7.50% | 3% | 7.50% |
| Solid particles of tanning substance | 5% | 5% | 5% | 5% | 3% | 2% | 1% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |
| Fragrance | Qs | qs | qs | qs | qs | qs | qs |

### Example 6

### Body Dry Oil

| | Examples | | | | | | |
|---|---|---|---|---|---|---|---|
| Component [% b.w.] | A | b | c | d | e | f | g |
| Lecithin or Phosphatidyl Choline | 2% | 2% | 3% | 3% | 4% | 5% | 2.50% |
| Sweet almond oil | 10% | 8% | — | 10% | — | — | 5% |
| Isopropyl palmitate | 10% | 5% | 5% | — | 7.50% | 5% | 10% |
| Squalane | 5% | 10% | — | 10% | 7% | 8% | 10% |
| Hydrogenated polydecenes | — | 5% | 14% | 7.50% | 7% | 10% | 10% |
| Synthetic esters | 5% | 15% | 20% | 7.50% | 15% | 20% | 8.50% |
| Dioctyl adipate | 10% | 10% | 5% | — | 7.50% | 5% | — |
| Cyclopentasiloxane (and) Cyclohexasiloxane | 8% | 20% | 10% | 15% | 25% | 10% | 20% |
| Phenyl Trimethicone | 30% | 10% | 20% | 25% | 15% | 20% | 30% |
| Cetearyl isononanoate | — | 10% | 10% | 5% | 5% | 10% | — |
| Botanical extract oils | 10% | — | 5% | — | 2% | 3% | — |
| PEG-8 laurate | 5% | — | 3% | 2% | 2% | 2% | 3% |
| Solid particles of tanning substance | 5% | 5% | 5% | 5% | 3% | 2% | 1% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |
| Fragrance | Qs | qs | qs | qs | qs | qs | qs |

### Example 7

### Silicone Gel

| | Examples | | | | | | |
|---|---|---|---|---|---|---|---|
| Component [% b.w.] | A | b | c | d | e | f | g |
| Hydrogenated Lecithin | 1.5% | 5% | 5% | 5% | 10% | 7.50% | 4% |
| Dicaprylyl ether | — | 5% | 15% | 15% | 10% | 7.50% | 20% |
| Glycerin | 40% | 20% | 20% | 18.5% | 20% | 25% | 33% |
| Butylene Glycol | 20% | 20% | 20% | 20% | 24.5% | 20% | 10% |
| Phenyl Trimethicone | 30% | 30% | 25% | 20% | 20% | 24% | 20% |
| Crosslinked elastomeric organopolysiloxanes | — | 5% | — | 10% | 4% | 8% | 6% |
| Silica Silylate | — | — | 5% | — | 3% | — | 1.5% |
| Silica | — | 5% | — | 2% | — | 2% | — |
| Bentonite | — | — | — | 1.50% | 2.5% | 2% | 1.5% |
| PPG-15 butyl ether | 3.5 | 5% | 5% | 4% | 3% | 2% | 3% |
| Solid particles of tanning substance | 5% | 5% | 5% | 5% | 3% | 2% | 1% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |
| Fragrance | Qs | qs | qs | qs | qs | qs | qs |

### Example 8

### Oil Spray

| | Examples | | | | | | |
|---|---|---|---|---|---|---|---|
| Component [% b.w.] | A | b | c | d | e | f | g |
| Hydrogenated polydecenes | 15% | 15% | 15% | 10% | 15% | 20% | 10% |
| C12-15 Alkyl benzoate (and) dipropylene | 15% | 20% | 20% | 10% | 5% | 15% | 22% |
| glycol dibenzoate (and) PPG-15 stearyl ether | | | | | | | |
| benzoate | | | | | | | |
| Cyclopentasiloxane (and) Cyclohexasiloxane | 20% | 20% | 20% | 28% | 15% | 17% | 15% |
| Isododecane | 15% | 20% | 20% | 5% | 11% | 10% | 10% |
| Synthetic, mineral or silicon waxes | 5% | 5% | — | 10% | 8% | 6% | 4.50% |
| Crosslinked elastomeric organopolysiloxanes | — | — | 5% | 5% | 5% | 5% | 5% |
| Isohexadecane | 15% | 10% | 10% | 10% | 7.50% | 10% | — |
| Isopropyl palmitate | — | 5% | 5% | — | 7.50% | — | 10 |
| Squalane | 5% | — | — | 10% | 15% | 5% | 7.50% |
| Synthetic esters | 5% | — | — | 7% | 8% | 10% | 15% |
| Solid particles of tanning substance | 5% | 5% | 5% | 5% | 3% | 2% | 1% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |
| Fragrance | Qs | qs | qs | qs | qs | qs | qs |

### Example 9

### Sunscreen Oil Gel

| | Examples | | | | | | |
|---|---|---|---|---|---|---|---|
| Component [% b.w.] | A | b | c | d | e | f | g |
| Hydrogenated polyisobutene | ad | ad | ad | ad | ad | ad | ad |
| | 100% | 100% | 100% | 100% | 100% | 100% | 100% |
| Isohexadecane | — | 7% | 5% | 8% | — | 10% | 5% |
| Mineral oil (an) hydrogenated | 35% | 32% | 20% | 18% | 28% | 25% | 15% |
| butylene/ethylene/styrene copolymer (and) | | | | | | | |
| hydrogenated ethylene/propylene/styrene | | | | | | | |
| copolymer | | | | | | | |
| Olefin/Styrene copolymer | — | — | 4% | 5% | 3% | 4% | 2% |
| Phenyl Trimethicone | — | 10% | 15% | 20% | 15% | 20% | 15% |
| Octyldodecyl Neopentanoate | 15% | 10% | 15% | 13% | 15% | 10% | 20% |
| Tocopheryl acetate | 0.5% | 0.5% | 1% | 0.50% | 0.50% | 1% | — |
| C12-15 alkyl benzoate | 14.5 | 10% | 11.5% | 10% | 5% | 4% | 4% |
| Bis-ethylhexyoxyphenol Methoxyphenyl | 2% | 2% | 3% | 4% | 2.50% | 1.50% | 4% |
| triazine | | | | | | | |
| Ethylhexyl Methoxycinnamate | 8% | 8% | 7% | 6% | 8.5% | 9% | 8% |
| Micronized (2-{4-[2-(4-Diethylamino-2- | 2% | 1% | 4% | | | | |
| hydroxy-benzoyl)-benzoyl]-piperazine-1- | | | | | | | |
| carbonyl}-phenyl)-(4-diethylamino-2-hydroxy- | | | | | | | |
| phenyl)-methanone | | | | | | | |
| Micronized 2,4,6-Tris(p-biphenylyl)-s-triazine | | | | 2% | 1% | 4% | |
| [CAS No. 31274-51-8] | | | | | | | |
| Avobenzone | | | | | | 1% | 2% |
| Octocrylene | | 1% | 1% | 4% | | | 4% |
| Tinosorb M (micronized CAS-Regno. | 3% | | | | 2% | | |
| 103597-45-1) | | | | | | | |
| CAS-Regno. 88122-99-0, Ethylhexyl triazone | | | | | | 3% | 1% |
| (Octyl triazone; Uvinul T 150) | | | | | | | |
| CAS-Regno. 118-60-5, 2-Ethylhexyl | | 1% | | | | | |
| salicylate | | | | | | | |
| Silica Silylate | — | — | 3% | 4% | 2% | 1% | — |
| Silica | 2% | 2.5% | — | — | — | — | 3% |
| Bentonite | — | — | 2.50% | 1.50% | — | — | — |
| Quaternized Bentonite | — | — | — | — | — | 1.50% | 3% |
| Silica Dimethyl Silylate | — | 3% | — | — | 3% | 2.50% | — |
| Solid particles of tanning substance | 5% | 5% | 5% | 5% | 3% | 2% | 1% |
| according to formula (II) or (III) or CAS | | | | | | | |
| Reg. No. 62147-49-3 or CAS Reg. No. 51795- | | | | | | | |
| 26-7 or CAS Reg. No. 675841-66-4 or CAS | | | | | | | |
| Reg. No. 62182-56-3 or CAS Reg. No. | | | | | | | |
| 757972-78-4. | | | | | | | |

What is claimed is:

1. A method for the colouration of skin or a keratinic fibre, characterized in that the skin or keratinic fibre is contacted with a formulation containing a dispersion of solid particles of a tanning substance selected from a compound of the formula I 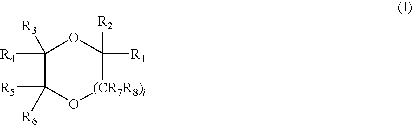
wherein i is 0 or 1 and each of R₁, R₂, R₃, R₄, R₅, R₆, R₇, R₈ independently of each other is selected from group consisting of hydrogen, OH, C₁-C₆alkyl, C₁-C₆hydroxyalkyl and C₂-C₆alkenyl; in an organic liquid of low polarity or non-polar organic liquid.

2. A method according to claim 1, wherein the organic liquid of low polarity or non-polar organic liquid is a silicon oil, a polysiloxane, a hydrocarbon-based oil or a fluoro oil or a mixture thereof.

3. A method according to claim 1, comprising additional compounds selected from the group consisting of fats, oils, allantoin, thickeners, water-free preservatives, perfume oils, antioxidants, UV absorbers, amino acids, soluble tanning substances, precursors or encapsulated forms of a self tanning substance, dispersing agents and penetration aids.

4. A method according to claim 1, wherein the particles are crystalline or amorphous and have an average diameter between the low micrometer and the nanoscale range.

5. A method according to claim 4, wherein 90% by weight or more of the particles are in the range between 10 micrometer and 1 nm.

6. A composition comprising
a) in an amount of 30-60% by weight of the composition a solid compound of the formula I according to claim 1, and
b) an organic liquid of low polarity or non-polar organic liquid.

7. A composition according to claim 6 comprising further components selected from the group consisting of fats, oils, allantoin, thickeners, water-free preservatives, perfume oils, antioxidants, UV absorbers, amino acids, soluble tanning substances, precursors or encapsulated forms of a self tanning substance, dispersing agents and penetration aids.

8. A composition according to claim 7, wherein the UV absorber is a micronized UV absorber.

9. A cosmetic formulation comprising
a) a solid particle of a tanning substance of a compound of the formula I according to claim 1 in dispersed form, and
b) a customary cosmetic carrier or auxiliary.

10. A cosmetic formulation according to claim 9, wherein component (a) is present in an amount of 1 to 15% by weight of the total cosmetic formulation.

11. A cosmetic formulation according to 9 in the form of a spray.

12. The use of a dispersion of solid particles of a tanning substance of a compound of the formula I according to claim 1 in an organic liquid of low polarity or non-polar organic liquid for the colouration of skin or a keratinic fibre, for the artificial tanning of the skin, for the levelling of differences in the brightness of the skin, for the protection of the skin against solar radiation, or for the colouration of human hair.

13. A process for the preparation of a cosmetic formulation for the artificial tanning of the skin or the colouration of a keratinic fibre, characterized in that a solid compound of the formula I according to claim 1 is subjected to wet-grinding, spray-drying, an expansion process using a supercritical fluid, or a reprecipitation, in an organic liquid of low polarity or non-polar organic liquid.
